(19) United States
(12) Patent Application Publication
(10) Pub. No.: US 2014/0018829 A1
(43) Pub. Date: Jan. 16, 2014

(54) SLIDING OVERHEAD CLIP AND ASSOCIATED METHODS

(71) Applicant: Paras Patani, New York, NY (US)

(72) Inventor: Paras Patani, New York, NY (US)

(73) Assignee: CASTLE SURGICAL, INC., New York, NY (US)

(21) Appl. No.: 13/981,864

(22) PCT Filed: Dec. 21, 2011
(86) PCT No.: PCT/US11/66575

§ 371 Date: Oct. 1, 2013

Related U.S. Application Data
(60) Provisional application No. 61/427,693, filed on Dec. 28, 2010.

(51) Int. Cl. A61B 17/08

(52) U.S. Cl. CPC A61B 17/083
USPC 606/151

(57) ABSTRACT

Sliding overhead clips and associated methods are disclosed that generally involve surgical clips with improved installation efficiency and higher forces for suture retention. The surgical clips include a first side and a second side, each of said sides having an upper body and a lower body. The surgical clips also generally include a flexible hinge integrally disposed between and joining the first side and second side. Further, the surgical clips include a first pair and second pair of female locking member and male locking member. The first pair of female and male locking members includes a first female locking member positioned on the first side and the corresponding first male locking member positioned on the second side. Additionally, the second pair of female and male locking members includes a second female locking member positioned on the second side and the corresponding second male locking member positioned on the first side.

### CROSS-REFERENCE TO RELATED APPLICATIONS

This application is based on and claims the priority benefit of U.S. Provisional Application No. 61/427,693, filed Dec. 28, 2010. The entire content of the foregoing provisional patent application is incorporated herein by reference.

### BACKGROUND

1. Technical Field

The present disclosure is directed generally to sliding overhead clips and associated methods and, more particularly, to surgical clips with improved installation efficiency, higher forces for suture retention, and advantageous distribution of suture forces over a larger surface area of the organ and/or tissue.

2. Background Art

Sutures are used in a variety of surgical settings, e.g., to pass through tissue and based on tied ends, to close surgical incisions. Although a surgeon may be able to tie the suture in exposed surgical areas, many surgical procedures are performed internally, for example, endoscopically and/or using minimally invasive surgical techniques, thereby making the suture inaccessible to the surgeon to tie by hand, thus complicating the surgical process. Further, because the organ and/or tissue operated on is generally soft, a risk that the suture can rip and/or damage the organ and/or tissue post-operation exists. Therefore, for example, kidney, liver, and/or other similar soft tissue surgery generally requires a surgical clip to distribute suture forces across a wider area of the organ and/or tissue, thus reducing the risk of ripping and/or damage. Suture clips of the prior art have been implemented in conjunction with suture knots in order to permit a surgeon to close surgical incisions with the suture or otherwise fix suture placement in inaccessible locations. In addition, suture clips have been introduced into surgical procedures to distribute the suture forces on the organ and/or tissue, thereby reducing the risk of damage post-operation.

However, the suture clips as taught by the prior art are poorly designed for suture retention and/or suture force bolstering applications. For example, existing surgical clips were primarily designed for vessel occlusion applications and over the years have been adapted for suture retention applications without properly addressing the unique requirements associated with suture retention, and vice versa. Moreover, in order to be applied, current surgical clips generally require the use of proprietary and expensive clip appliers designed by their respective manufacturers. (See, e.g., U.S. Pat. No. 5,234,449 to Bruker et al. and U.S. Patent Publication No. 2006/0217749 to Wilson et al.) However, as medical and surgical technology has changed, the existing surgical clips and their respective clip appliers have only experienced minor modifications. Limitations associated with current clips used in suture retention applications increase the likelihood of surgical complications that can result in significant expenses for hospitals, inconvenience for surgeons, and compromises in patient care. Indeed, patient complications sometimes arise, causing morbidity and/or requiring emergency surgery to remove the clips many months after the initial operation. In addition, the FDA has advised multiple recalls of the prior art clips due to significant patient complications. However, surgeons continue to implement these clips and, thus, compromise patient care, since no improved alternative exists.

For example, robotic surgical procedures usually require the use of one to two, 12 mm diameter access trocar ports for an endoscopic camera and two to three, 8 mm diameter access trocar ports for the surgery itself. However, the previously mentioned clip appliers generally have diameters of 10 mm or 11 mm, making them too large to be used with the 8 mm diameter trocar access ports. Therefore, in order to apply the requisite surgical clips in robotic applications, surgeons must undertake one of the following alternatives: (i) insert an additional 10 mm or 12 mm trocar port specifically for the clip applier (highly unlikely surgical approach in view of greater patient trauma), (ii) remove the camera and utilize its trocar port for the clip applier (inconvenient), or (iii) remove one of the 8 mm trocar ports and insert a 10 mm trocar by making the 8 mm incision larger (inconvenient and also introduces greater patient trauma). The inconveniences associated with the third option become further exacerbated when surgery requires a subsequent re-introduction of the 8 mm port in what has become a 10 mm incision in the patient's abdomen, thereby compromising insufflation.

From a financial perspective, clip appliers for surgical clips of the prior art are relatively expensive and can cost upwards of $2,400 each. They are typically designed to be reused ten times prior to their replacement and generally require an additional cost for sterilization after each use (e.g., a resterilization cost on the order of $300). In addition, typical operating room rates for robotic surgery are charged at approximately $90 per minute. A robotic prostatectomy procedure, for example, is often completed in less than 20 minutes. Unnecessarily wasting valuable minutes to reconfigure and reinsert trocar ports, as required by the existing clip appliers, increases the costs of surgery significantly. Longer operating room times further mean longer anesthesia time and greater amounts of anesthesia for the patient.

In view of these issues, at least one medical device manufacturer recently adapted its clip applier design for use with 8 mm trocar access ports in robotic surgery. Although this modification adequately addressed the concerns regarding the mismatch of clip applier diameters identified above, in making the modification, the manufacturer more significantly increased its pricing, thereby further increasing the cost of surgery.

In addition, the use of proprietary clip appliers requires hospitals to hold expensive inventory and maintain sterile, secure and traceable FDA-regulated processes for proper biohazard removal, transportation and sterilization, further adding associated costs, administrative difficulties and potential health risks to staff.

With regard to human factors of design and usability, the existing clip appliers are cumbersome for surgeons to use. For example, in a partial nephrectomy procedure, suturing and closure of the renal cavity is conducted by pulling a suture taught anteriorly (up away from the front surface of the patient) with one hand while applying the corresponding surgical clip medially (from the side) with the other hand. In addition, because two surgical clips are applied successively, the surgeon is generally required to pull up on the suture while applying and holding down the first surgical clip, while an assistant applies the second surgical clip in order to fixate the first surgical clip in place. Further, accurate positioning of the surgical clip is crucial to lower risks of future complications. Therefore, most surgeons require a surgical assistant to deliver and apply the surgical clips during the procedure, adding to the cost of the procedure. Moreover, this part of the surgical procedure requires precise communication with the assistant and may lead to errors in an already complex and important part of surgery. Hence, many surgeons find this aspect of the procedure extraordinarily frustrating. This frustration is exacerbated by the fact that each surgical procedure often requires application of about 12 to 30 surgical clips.

A further limitation of surgical clips as taught by the prior art is with respect to the types of sutures utilized during surgery. Generally, sutures are available in a variety of forms, i.e., monofilament, braided and the like. Specifically, monofilament sutures are composed of a single filament and generally are more likely to slip or pass through a clip. On the other hand, braided sutures are composed of two or more filaments and the braided surfaces generate greater friction when interacting with tissue, clips, and/or other structures. Thus, braided sutures generally reduce the amount of slip or passage through a clip as compared to monofilament sutures. However, due to the roughness of the surface of the braided suture, greater inflammation and damage to tissue generally results as the braided suture is passed through the tissue. Therefore, although surgeons generally prefer the utilization of the monofilament suture, the braided suture is typically utilized due to the risk of clip slippage in a wet surgical environment.

The surgical clips of the prior art are typically utilized only with braided sutures so as to gain the increased frictional forces, due at least in part to the lack of a force high enough to securely hold the suture in place. Therefore, more tissue damage, and thereby prolonged recuperation periods, are likely to occur from the use of braided suture in conjunction with the clips as taught by the prior art.

Thus, a need exists for surgical suture clips which provide better suture retention, address the root causes of patient complications due to prior art clips, provide a viable and superior alternative to surgeons and/or potentially help improve patient outcomes. A further need exists for limiting or potentially reducing costs to administer surgical suture clips, thereby increasing the efficiency in cost and installation. Still further, a need exists for a suture clip that is capable of providing a high enough engagement force relative to a suture so as to prevent slippage and/or passage of a monofilament suture through the clip, thereby permitting the use of either a monofilament or braided suture, as desired by the surgeon.

These and other needs are addressed by the sliding overhead clips and associated methods of the present disclosure.

### SUMMARY

In accordance with embodiments of the present disclosure, sliding overhead clips and associated methods are disclosed that generally involve surgical clips with improved installation efficiency and higher forces for suture retention. Moreover, the disclosed surgical clips may be advantageously employed to engage both braided and monofilament sutures, thereby enhancing the utility and efficacy of the disclosed clips in a range of surgical procedures. Indeed, exemplary embodiments of the present disclosure provide surgical clips that may be applied to either braided or monofilament suture threads; once applied to the suture thread, the disclosed clips permit sliding engagement in a first direction relative to the suture thread, but substantially prevent movement relative to the suture thread in the opposite direction.

An exemplary surgical clip as disclosed herein includes a first leg or side and a second leg or side, each of said legs/sides having an upper body and a lower body. The surgical clip also generally includes a flexible hinge integrally disposed between and joining the first leg/side and second leg/side. Further, the surgical clip generally includes a first pair and second pair of cooperating locking members. The first and second cooperating locking members may each advantageously take the form of cooperating male/female locking members, e.g., cooperating clasp and backstop clasp. In exemplary implementations, the first pair of female and male locking members includes a first female locking member formed or positioned on or relative to the first leg/side, and a corresponding first male locking member formed or positioned on or relative to the second leg/side. Additionally, the second pair of female and male locking members may advantageously include a second female locking member formed or positioned on or relative to the second leg/side and a corresponding second male locking member formed or positioned on or relative to the first leg/side.

In accordance with another exemplary embodiment of the present disclosure, a method for surgical clip application is provided. The disclosed method generally involves introducing a surgical clip fabricated according to the present disclosure into a surgical environment and hooking or otherwise engaging the surgical clip relative to a braided or monofilament suture. The exemplary method of surgical clip application also includes securing the first leg/side of the surgical clip relative to the second leg/side of the surgical clip through cooperative structural interaction between cooperative locking members, and sliding the surgical clip along the suture relative to a tissue surface. Of note, the disclosed surgical clip may include structural feature(s) that permit sliding motion relative to the suture in a first direction, i.e., toward the tissue surface, but substantially prevent sliding motion in the opposite direction. The foregoing structural feature(s) are particularly advantageous when the disclosed surgical clip is applied to a monofilament suture.

In accordance with yet another embodiment of the present disclosure, a method for surgical clip application is provided. The disclosed method generally involves positioning a surgical clip fabricated according to the present disclosure in an open position such that a suture is positioned between the first leg and the second leg of the surgical clip. The disclosed method generally further includes securing the suture between the first leg and the second leg of the surgical clip by moving the first leg and the second leg from the open position into close approximation, thereby causing interlocking engagement of the first cooperating locking members and interlocking engagement of the second cooperating locking members, and introducing the surgical clip into the surgical environment.

The first leg/side and second leg/side of the surgical clip utilized in the disclosed methods each generally include or define an upper body and a lower body. The surgical clip also generally includes a flexible hinge which is integrally disposed between and joins the first leg/side and second leg/side. Further, the disclosed surgical clip typically includes a first and second pair of cooperating locking members, e.g., cooperative female and male locking members.

The exemplary sliding overhead clip is generally designed to fit through an 8 mm or larger trocar access port without the need for a proprietary clip applier. However, the surgical clip of the present disclosure may be dimensioned for utility in alternative surgical environments, as will be readily apparent to persons skilled in the art. During conventional minimally invasive surgical procedures, surgeons are already inserting multiple 8 mm (or larger) trocar ports around the patient's abdomen in order to perform the surgery. Of note, for minimally invasive nephrectomy procedures, the noted ports are generally placed exactly where they need to be in order for a surgeon to access the clip application areas for placement of the disclosed surgical clips relative to suture. Therefore, utilizing these existing trocar ports in conventional nephrectomy procedures to insert and apply the exemplary sliding overhead clip of the present disclosure imposes no additional incremental cost, time or port position modifications, and may actually provide an overall cost reduction to the hospital.

Furthermore, during surgical procedures, standard endoscopic forceps are used routinely for tissue suturing, mobilization and retraction. To accommodate this, footpads and upper body features may be incorporated into the design of exemplary sliding overhead clips of the present disclosure. The footpads allow a surgeon to actuate and apply the sliding overhead clip to the upper body via standard forceps, i.e., endoscopic forceps, instead of clip appliers. The use of standard endoscopic forceps reduces the material application, sterilization, inventory and management costs generally incurred during surgeries involving clip placement. Thus, the sliding overhead clip and its actuation via standard endoscopic forceps also imposes no additional incremental cost, time or port position modifications, and may further provide an overall cost reduction to the hospital.

In further embodiments of the present disclosure, the inclusion of two pairs of cooperating locking members, e.g., two pairs of clasps and backstop clasps, in the design/operation of the disclosed sliding overhead clip provides enhanced security to reduce the risk of the clip opening or otherwise loosening its grip on a suture post-operation. The disclosed clip may also define at least one recess that functions to facilitate quick hooking/engagement and sliding of the clip down a length of suture to a desired position, e.g., a position proximate a tissue surface. Clip actuation from overhead, instead of medially, enables the surgeon to apply the clip autonomously without requiring surgical assistance, thereby reducing the costs of surgery. The design of the exemplary clip permits precise placement and security of the suture in the center of the clip, ensuring a substantially evenly distributed suture force over the tissue surface when the clip is slid to a position proximate the tissue surface. As additional security, the recess and/or recesses defined by the suture clips of the present disclosure also enable multiple clips to be applied in close approximation to each other on a suture, e.g., in a “stacked” or “nested” orientation, if desired by the surgeon.

Thus, the exemplary embodiments of the sliding overhead clip disclosed herein meet the needs associated with suture clips that are not met by suture clips of the prior art. Specifically, the exemplary sliding overhead clips of the present disclosure provide significantly improved suture retention (both when used with braided and monofilament sutures), reduce the costs associated with administering suture clips (including potential application with conventional forceps and reduction of the number of clips implemented), increase the efficiency and efficacy in installation of suture clips relative to suture, and improve the distribution of suture forces relative to an organ and/or tissue.

Additional features, functions and benefits associated with the disclosed sliding overhead clip and associated methods will be apparent from the detailed description which follows.

### BRIEF DESCRIPTION OF THE DRAWINGS

For a fuller understanding of the present disclosure, reference is made to the following description taken in connection with the accompanying drawings, in which:

FIG. 1A is a view of sliding clip techniques currently implemented by the prior art;

FIGS. 1B and 1C is a view of exemplary clip appliers currently implemented by the prior art;

FIG. 2 is an isometric top view of the back of the exemplary sliding overhead clip in an open position in accordance with the present disclosure;

FIG. 3 is an isometric bottom view of the back of an exemplary sliding overhead clip in an open position;

FIG. 4 is an isometric top view of the front of an exemplary sliding overhead clip in an open position;

FIG. 5 is an isometric bottom view of the front of an exemplary sliding overhead clip in an open position;

FIG. 6 is an orthographic view of the male side of an exemplary sliding overhead clip in a closed position;

FIG. 7 is an orthographic view of the hinge of an exemplary sliding overhead clip in a closed position;

FIG. 8 is an orthographic view of the side opposite the hinge of an exemplary sliding overhead clip in a closed position;

FIG. 9 is an orthographic view of the female side of an exemplary sliding overhead clip in a closed position;

FIG. 10 is an isometric top view of an exemplary sliding overhead clip in a closed position;

FIG. 11 is an isometric bottom view of an exemplary sliding overhead clip in an open position;

FIG. 12 is a top view of an exemplary sliding overhead clip in a closed position;

FIG. 13 is a bottom view of an exemplary sliding overhead clip in a closed position depicting recesses that accommodate stacking/nesting of additional sliding overhead clips;

FIG. 14 is an isometric top view of the back of an exemplary sliding overhead clip in an open position with suture and forceps in position to actuate the exemplary sliding overhead clip;

FIG. 15 is an isometric top view of the back of an exemplary sliding overhead clip in a closed position with suture and forceps in position;

FIG. 16 is an isometric top view of the back of an exemplary sliding overhead clip in an open position with grasping ridges;

FIG. 17 is an orthographic view of the hinge of an exemplary sliding overhead clip in an open position with grasping ridges;

FIG. 18 is a top view of an exemplary sliding overhead clip in an open position with grasping ridges;

FIG. 19 is an orthographic view of the male side of an exemplary sliding overhead clip in an open position with grasping ridges;

FIG. 20 is a cross-sectional perspective view of the top of an exemplary sliding overhead clip in a closed position;

FIG. 21 is a cross-sectional perspective view of the bottom of an exemplary sliding overhead clip in an open position;

FIG. 22 is a detailed perspective view of a male hinge side of an exemplary sliding overhead clip;

FIG. 23 is a detailed perspective view of a male side opposite of the hinge of an exemplary sliding overhead clip;

FIG. 24 is a detailed top view of a male side of an exemplary sliding overhead clip;

FIG. 25 is a detailed bottom view of a male side of an exemplary sliding overhead clip;

FIG. 26 is a detailed perspective view of a female hinge side of an exemplary sliding overhead clip;

FIG. 27 is a detailed perspective view of a female side opposite of the hinge of an exemplary sliding overhead clip;

FIG. 28 is a detailed top view of a female side of an exemplary sliding overhead clip;

FIG. 29 is a detailed bottom view of a female side of an exemplary sliding overhead clip;

FIG. 30 is a detailed view of ridges formed on a female side of an exemplary sliding overhead slip;

FIG. 31 is a detailed view of ridges formed on a male side of an exemplary sliding overhead clip;

FIG. 32 is a detailed view of anti-backup features formed on a male side of an exemplary sliding overhead clip;

FIG. 33 is a perspective top view of another exemplary embodiment of a sliding overhead clip, including grip point recesses;

FIG. 34 is a perspective bottom view of another exemplary embodiment of a sliding overhead clip, including grip points;

FIG. 35 is a perspective top view of another exemplary embodiment of the hinge of a sliding overhead clip;

FIG. 36 is a perspective bottom view of another exemplary embodiment of the hinge of a sliding overhead clip;

FIG. 37 is a top view of another exemplary embodiment of the hinge of a sliding overhead clip; and

FIG. 38 is a chart comparing strength profiles for an exemplary sliding overhead clip of the present disclosure and clips as taught by the prior art.

### DESCRIPTION OF EXEMPLARY EMBODIMENT(S)

In accordance with embodiments of the present disclosure, sliding overhead clips and associated methods are disclosed that generally involve surgical clips with improved installation efficiency and higher forces for suture retention. An exemplary surgical clip as disclosed herein includes a first leg/side and a second leg/side, each of said legs/sides defining an upper body and a lower body. The surgical clip also generally includes a flexible hinge integrally disposed between and joining the first leg/side and second leg/side. Further, the disclosed surgical clip generally includes a first pair and second pair of cooperating locking members, e.g., cooperating female locking member and male locking member. The cooperating locking members may advantageously take the form of a clasp and backstop clasp. The first pair of female and male locking members typically includes a first female locking member formed or positioned on the first leg/side and a corresponding first male locking member formed or positioned on the second leg/side. Additionally, the second pair of female and male locking members typically includes a second female locking member formed or positioned on the second leg/side and a corresponding second male locking member formed or positioned on the first leg/side.

In accordance with another embodiment of the present disclosure, a method for surgical clip application is provided. The disclosed method generally involves introducing a surgical clip of the present disclosure into a surgical environment and hooking or engaging the surgical clip relative to a suture (braided or monofilament). The exemplary method of surgical clip application also includes securing the first leg/side of the surgical clip relative to the second leg/side of the surgical clip and sliding the surgical clip along the suture into a desired position, e.g., a position proximate a tissue surface. In addition, once the surgical clip has been hooked and/or engaged relative to a suture, the exemplary method for surgical clip application can also generally include initially pulling up on the suture while sliding the surgical clip posteriorly along and/or down the suture into a desired position, e.g., a position proximate a tissue surface, and subsequently securing the first leg/side of the surgical clip relative to the second leg/side of the surgical clip in order to maximize arterial pressure on the organ. Thus, after the first leg/side and second leg/side of the surgical clip have been secured, the surgical clip can optionally be slid further into a desired position, e.g., a position proximate a tissue surface. The first leg/side and second leg/side of the surgical clip each generally have or define an upper body and a lower body. The surgical clip also typically includes a flexible hinge (e.g., a living hinge) which is integrally disposed between and joins the first leg/side and leg/second side. Still further, the surgical clip includes a first and second pair of cooperating locking members, e.g., first and second female and male locking members. In use, the surgical clip is applied to a suture and slid to a desired position, e.g., a position proximate a tissue surface. Of note, the surgical clip may include structural feature(s) that permit sliding motion relative to a suture in a first direction, but substantially prevent motion of the surgical clip relative to the suture in the opposite direction. Although a first surgical clip may be implemented in conjunction and opposite to a second surgical clip in order to maximize arterial pressure, the exemplary surgical clip may also be implemented as a single unit if desired. Further, a suture knot may be tied anteriorly and/or posteriorly to the surgical clip, a first surgical clip may be actuated/closed around the suture prior to insertion of the suture into the surgical environment and a second surgical clip may be actuated/closed around the opposite portion of the suture to provide the desired pressure, and a suture knot may be tied around the surgical clip subsequent to the actuation/closing of the surgical clip around the suture.

In accordance with yet another embodiment of the present disclosure, a method for surgical clip application is provided. The disclosed method generally involves positioning a surgical clip fabricated according to the present disclosure in an open position such that a suture is positioned between the first leg and the second leg of the surgical clip. The disclosed method generally further includes securing the suture between the first leg and the second leg of the surgical clip by moving the first leg and the second leg from the open position into close approximation, thereby causing interlocking engagement of the first cooperating locking members and interlocking engagement of the second cooperating locking members, and introducing the surgical clip into the surgical environment.

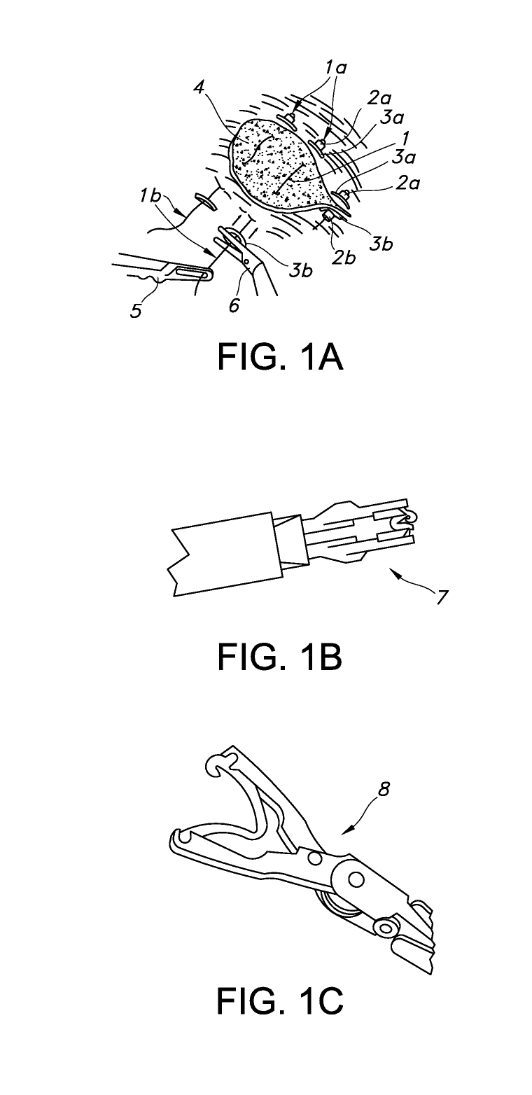

With reference to FIG. 1A, an exemplary current sliding clip technique is depicted as implemented by the prior art. Upon completion of, for example, a tumor excision, the suturing and closure of the renal cavity 4 generally commences by extra-corporeally securing a Lapra-Ty™ clip 2a and a Hem-O-Lok™ clip 3a onto a distal end 1a of the suture 1 and tying a knot at the distal end 1a of the suture 1 adjacent to the Lapra-Ty™ clip 2a. The proximal end 1b of the suture 1 is then generally inserted through a trocar and through the organ, a procedure which increases the risk of introducing infection and subsequent necrosis intra-corporeally due to the initial extra-corporeally completed process. The suture 1 is subsequently drawn through the renal cavity 4 and held up using endoscopic forceps through a trocar port, thereby tightening the Lapra-Ty™ clip 2a and a Hem-O-Lok™ clip 3a on the distal end 1a of the suture 1 against the rental cavity 4. The surgeon then generally applies a Hem-O-Lok™ clip 3b intra-corporeally via the endoscopic clip appliers through an additional trocar port and secures the Hem-O-Lok™ clip 3b on the suture 1 while continuing to hold up the suture 1 with the other hand. It should be noted that the introduction and application of each clip, i.e., Hem-O-Lok™ and Lapra-Ty™ clips, requires the use of specific clip appliers configured to lock each clip. In particular, FIGS. 1B and 1C depict exemplary clip appliers 7 and 8 as currently utilized in the prior art. The surgeon further passes the endoscopic clip appliers to the assistant and, using a second endoscopic forceps 6, pushes the Hem-O-Lok™ clip 3b down the suture 1 and onto the organ, typically pulling up on the suture 1 as much as possible so as to maximize arterial pressure and sufficiently close the rental cavity 4. The low grasping pressure of the Hem-O-Lok™ clip 3b permits the Hem-O-Lok™ clip 3b to slide relatively easily along and down the suture 1. Further, in order to maintain the maximum arterial pressure desired, while the surgeon holds the position of the Hem-O-Lok™ clip 3b, the surgical assistant, using the Lapra-Ty™ clip 2b appliers through yet another additional trocar port, generally fixates the Lapra-Ty™ clip 2b anteriorly to the Hem-O-Lok™ clip 3b. A knot is then tied intra-corporeally on the proximal end 1b of the suture 1 anteriorly to the Lapra-Ty™ clip 2b.

As is understood by those of ordinary skill in the art, an interrupted stitch, rather than a running stitch, is typically implemented to, e.g., reduce the risk of patient complications if one of the stitches breaks. Thus, the above-described process with respect to FIG. 1A is generally repeated for each interrupted stitch, thereby utilizing at least one interrupted stitch for about each 1 cm length of incision. For example, the average tumor diameter and the corresponding tumor cavity at the time of diagnosis is generally about 2.6 cm, requiring about twelve (12) surgical clips, e.g., about 6 Lapra-Ty™ clips and about 6 Hem-O-Lok™ clips. Similarly, rental tumors are generally about 4 cm or smaller in diameter, requiring about sixteen (16) surgical clips, e.g., about 8 Lapra-Ty™ clips and about 8 Hem-O-Lok™ clips. Partial nephrectomy is regularly performed for tumor diameters up to about 7 cm, requiring about twenty-eight (28) surgical clips, has been implemented for tumors of about 11 cm in diameter, requiring about forty-four (44) surgical clips, and similar surgical procedures have been utilized for organs larger than the kidney, e.g., the liver. Thus, the large amount of surgical clips and repeated procedures implemented as taught by the prior art, along with the additional equipment and assistants required, result in a cumbersome and inefficient process with respect to both costs and time.

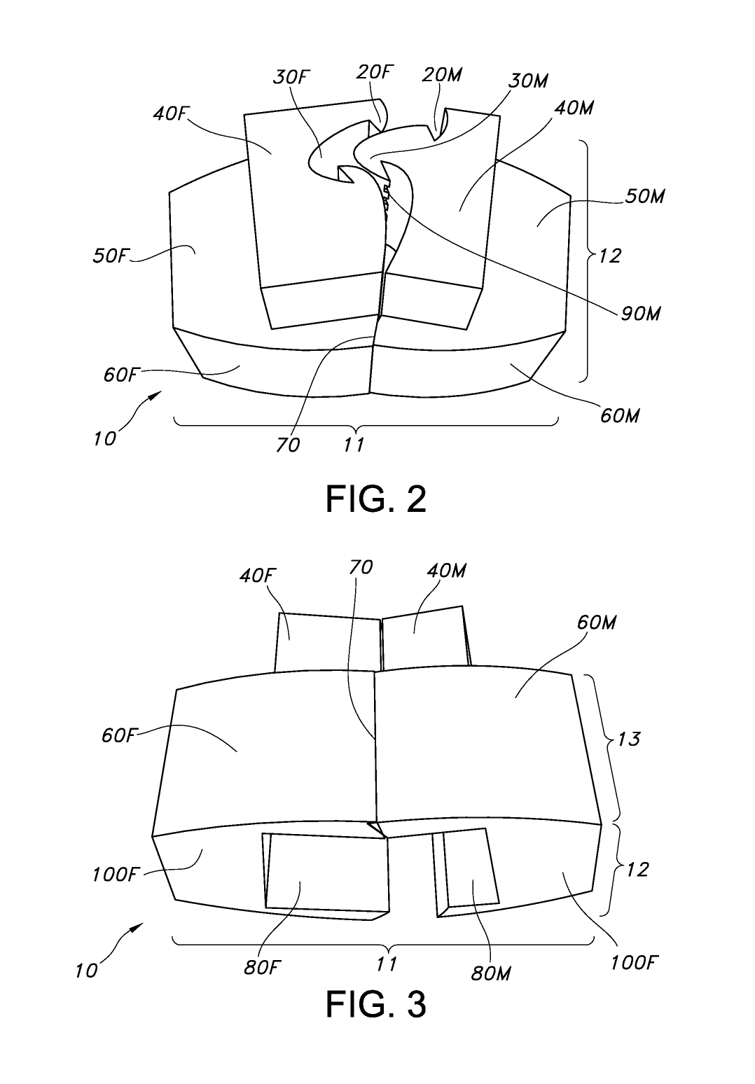

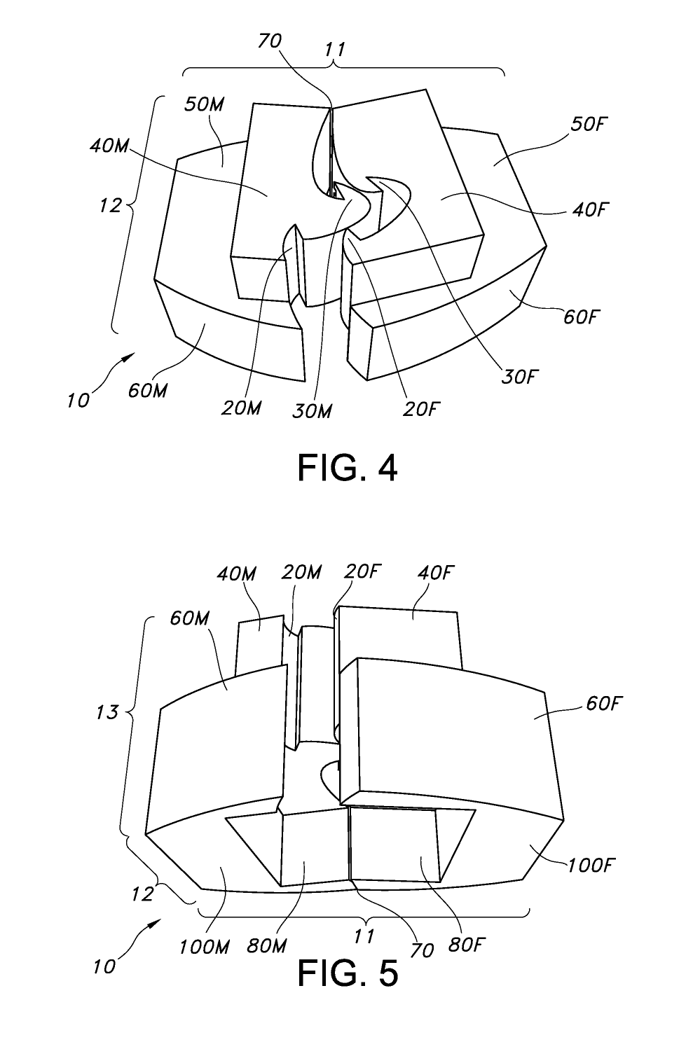

Turning now to FIGS. 2-5, an exemplary embodiment of a sliding overhead clip 10 (hereinafter “clip 10”) is depicted in accordance with the present disclosure. It should be noted that in the discussion of the Figures, reference characters specific to the “female” side shall be designated with an “F” and reference characters specific to the “male” side shall be designated with an “M”, e.g., upper body 40F and 40M, respectively. However, if reference is being made to the corresponding components of the “female” side and the “male” side as a pair, the “F” and “M” designation may not be utilized, e.g., upper body 40. Still with reference to FIGS. 2-5, clip 10 is depicted in its standard open position, i.e., the position in which the surgeon would receive the clip 10 when the package is opened or the clip is otherwise delivered to the sterile field. FIGS. 2 and 3 depict the top and bottom views of clip 10 from the hinge 70 side, while FIGS. 4 and 5 depict the top and bottom views of clip 10 from a front side (i.e., a side opposite hinge 70).

Clip 10 may be manufactured from a single, unibody piece of material, e.g., a biocompatible plastic (biopolymer) or other biologically acceptable material implemented in the art. In addition, the present clip can be made from any of various absorbable or non-absorbable biologically compatible materials, including, but not limited to, homopolymer or copolymer of polylactic acid and polyglycolic acid, caprolactone, polydioxanone, polytetrafluoroethylene, nylon, polyethylene, titanium, tantalum, or the like. Preferred materials for use in fabricating the disclosed clip do not disrupt post-operative or other subsequent diagnostic procedures used on the patient, i.e., X-ray imaging, CAT scanning, and the like. Clip 10 may be fabricated using one of the above biomaterials by means of conventional polymeric additive, subtractive, injection or extrusion methods for rapid prototyping or production. Upon fabrication, clip 10 may undergo any of various dimensional stabilization methods known in the art, i.e., scouring, annealing, crystallization and the like. Finally, clip 10 may be rendered sterile by any of well-known sterilizable procedures, i.e., ethylene-oxide, cobalt irradiation or other similar processes, depending on the specific biocompatible material used.

Still with reference to FIGS. 2-5, clip 10 includes two legs/sides, the “female” side 10F and the “male” side IOM, which interlock with each other at backstop clasp 20 and clasp 30 when clip closure is actuated and pressed together, e.g., applying a closure force with forceps. As can be seen in FIG. 2, clasp 30M further includes ridges 90M for an improved hold of a suture. As will be discussed in greater detail below, at least one of clasps 30M and 30F (generally both) include ridges 90M and 90F, respectively, which interlock with each other, forming a tongue and groove joint 90FM and establishing a strong hold of the suture.

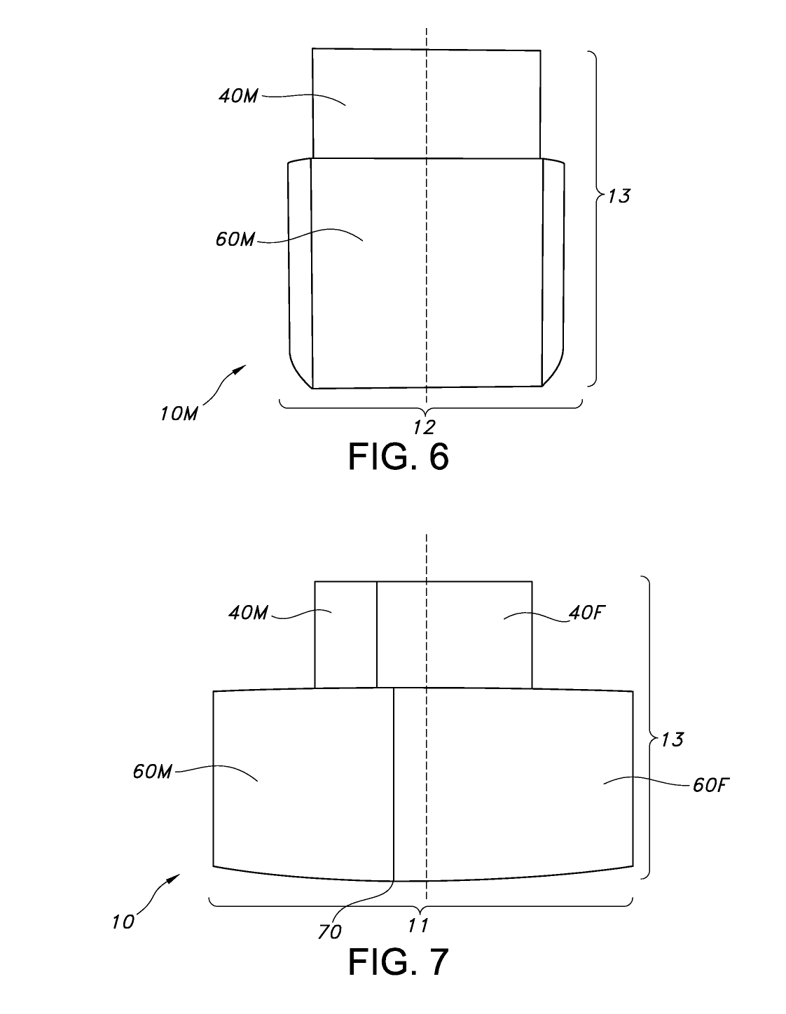

Clip 10 has outer dimensions designated by width 11, depth 12 and height 13, which are specifically designed to provide the largest clip dimensions possible, while still fitting through a desired port/cannula, e.g., an 8 mm or larger laparoscopic trocar access port. However, as would be apparent to those of ordinary skill in the art, the clip 10 disclosed herein may be of varying configurations and dimensions such that it can be introduced into the surgical area through a smaller laparoscopic trocar access port, e.g., a 5 mm port, and/or used in open surgical procedures. Similar figures showing clip 10 in its closed position will be discussed below with respect to FIGS. 6-13.

With further reference to FIGS. 2-5, each leg/side of the clip 10 includes or defines an upper body 40, a forceps footpad 50, a lower body 60, a recessed underside 80, and a foundation 100. The legs/sides of clip 10 are joined by hinge 70, which typically relies on the elasticity of the material of construction, e.g., the biomaterial, for its flexure. Specifically, hinge 70 presents a simple flexure point for the “female” and “male” legs/sides of clip 10. During actuation of clip 10, which actuation force may be applied via forceps, hinge 70 is stressed and remains in a stressed position upon closure of clip 10 throughout the patient's wound-healing period. Generally, the hinge 70 and/or the hinge section of clip 10 is resilient, exhibits elastic memory and allows for proper handling, actuation and placement of clip 10 relative to a suture (monofilament and/or braided) according to the present disclosure.

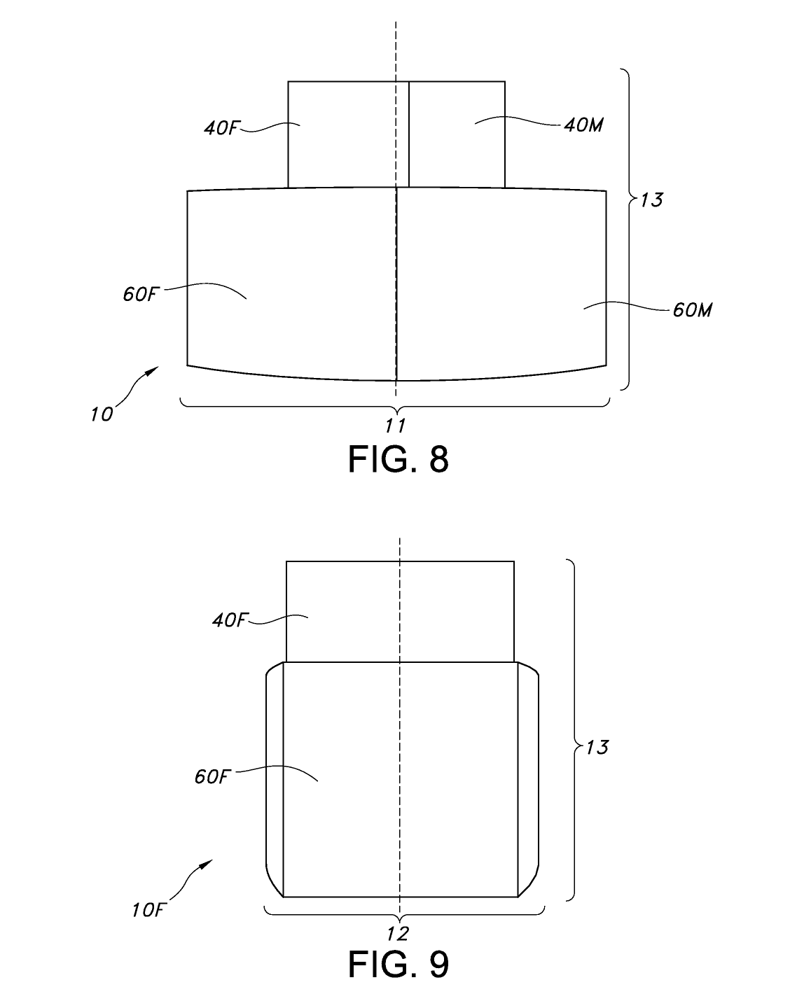

Turning now to FIGS. 6-9, orthographic views of the exemplary clip 10 are provided in a closed position. Specifically, FIG. 6 is a view of the “male” leg/side 10M of the exemplary clip 10, from which can be seen the upper body 40M and the lower body 60M. FIG. 7 is a side view of the hinge 70 of clip 10 in a closed position. The upper body 40 and lower body 60 can be seen again from both the “female” side 10F and “male” side 10M. FIG. 8 represents a view of the side of clip 10 opposite hinge 70 in a closed position, including upper body 40 and lower body 60 for both the “female” leg/side 10F and “male” leg/side 10M. Finally, FIG. 9 is a view of the “female” leg/side 10F of clip 10 in a closed position, including the upper body 40F and lower body 60F.

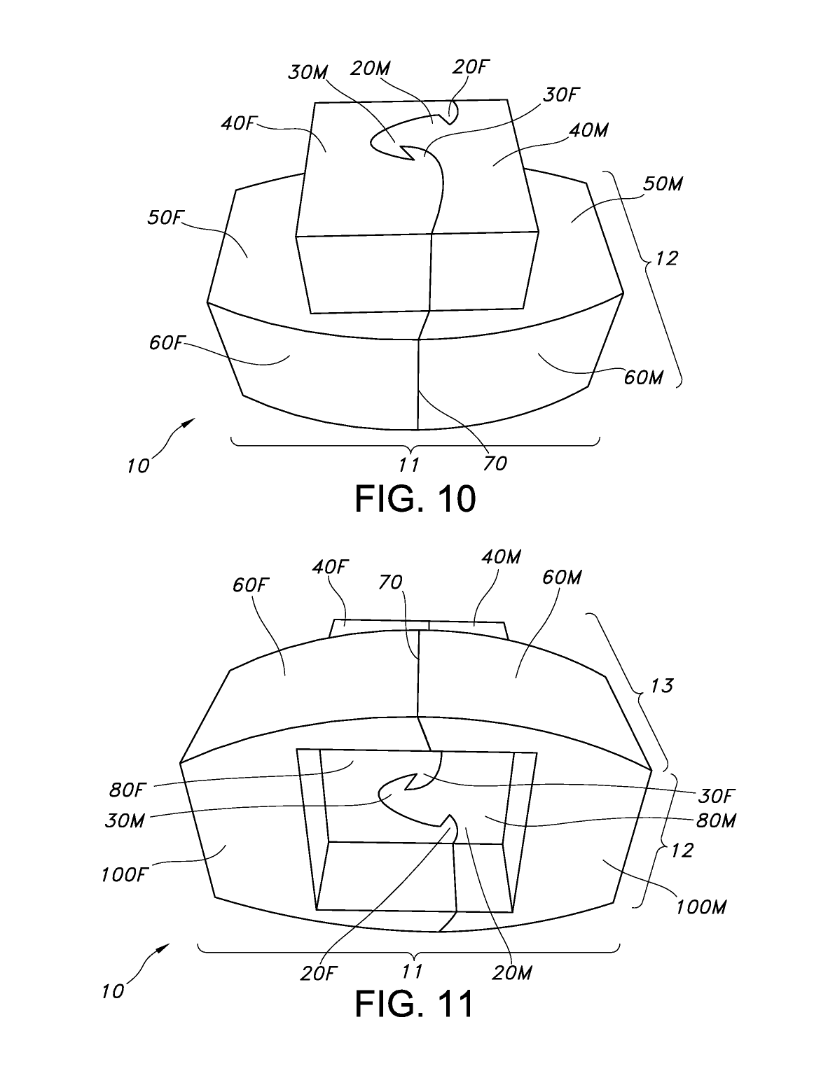

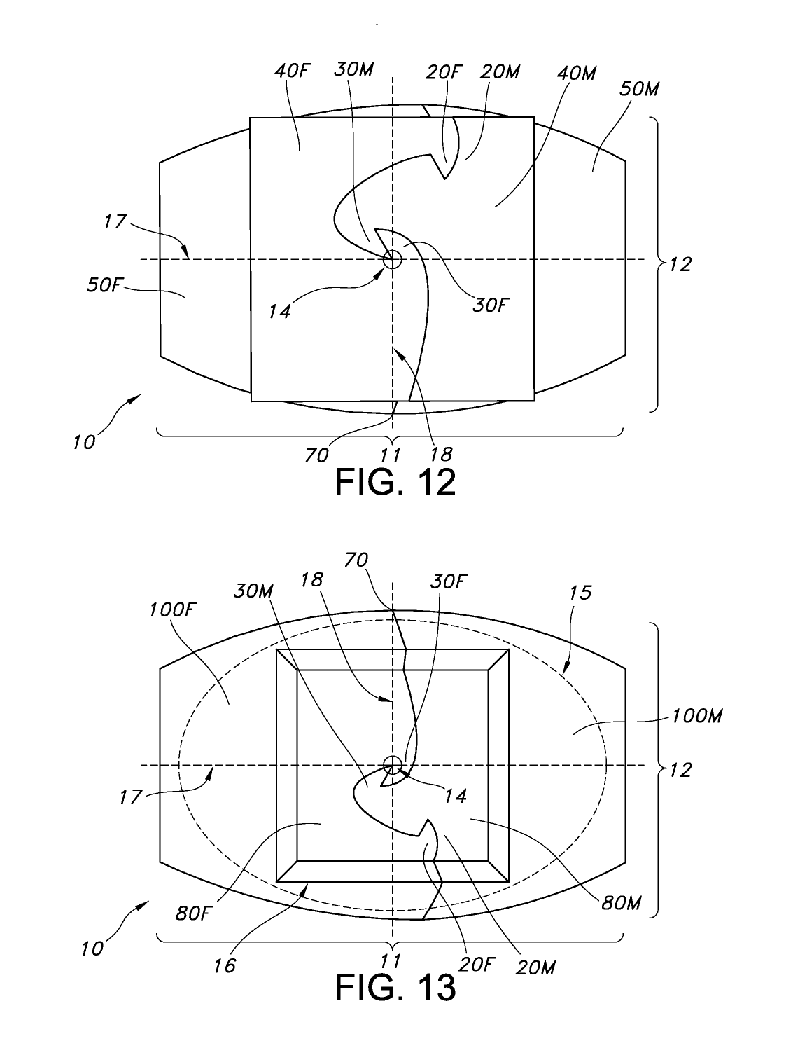

With reference to FIGS. 10-13, perspective top and bottom views of the exemplary clip 10 are depicted in a closed position. During actuation of clip 10, clasp 30 and backstop clasp 20 engage and come together to hold a piece of suture in the region, e.g., crevice, between clasp 30M and 30F. Specifically, as can be seen in FIGS. 12 and 13, a suture would be held substantially in the center of the clip 10, i.e., the region/crevice between clasp 30M and 30F, which is depicted as center 14.

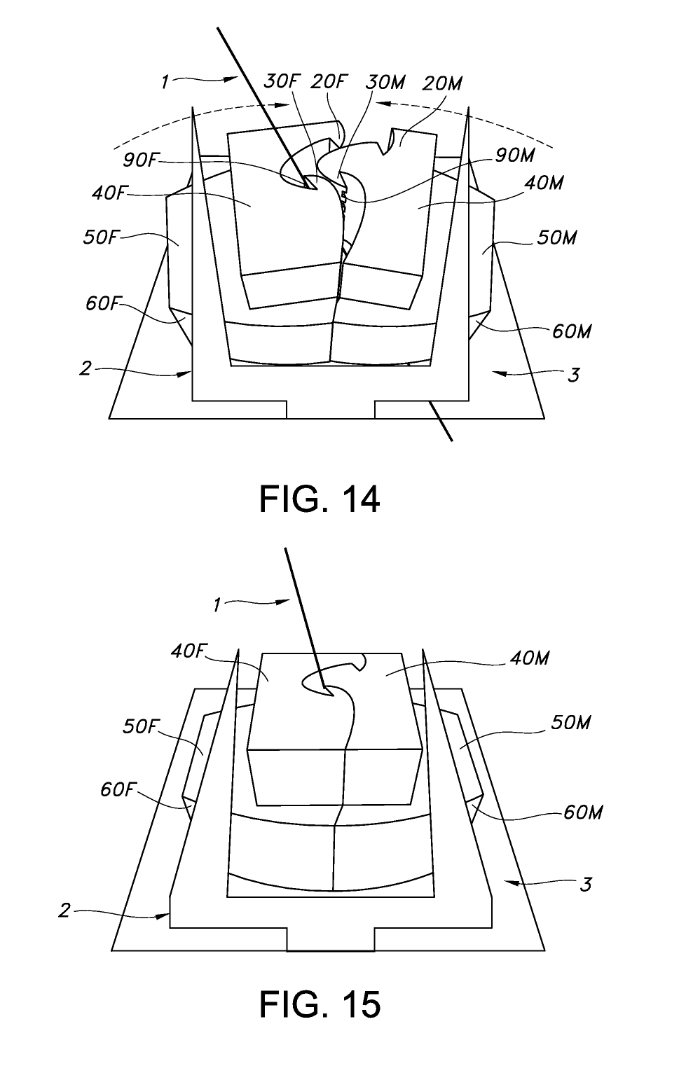

Now with reference to FIGS. 14 and 15, the open and closed positions of the exemplary clip 10 are depicted, including the suture 1 and forceps 2 in position to actuate the exemplary clip 10. Clip 10 has three primary features designed to assist a surgeon during application of clip 10. As can be seen in FIGS. 14 and 15, clip 10 includes a “sliding mechanism” on clasp 30F by which clip 10 can be inserted medially through a trocar port, hooked or otherwise engaged relative to suture 1 and easily slid down the length of suture I to a desired position, e.g., a position proximate tissue surface 3 of the organ or body of tissue being sutured. Clip 10 further has footpads 50F and 50M on the lower body portions 60F and 60M, respectively, for structural interaction with the jaws of endoscopic forceps 2. Thus, clip 10 may be actuated via forceps 2 looking posteriorly from overhead. Further, clip 10 provides secure holding, fixation and retention by means of clasp 30 and backstop clasp 20. Specifically, during actuation, clasps 30F and 30M come together to hold suture 1 in the crevice of clasp 30F, i.e., center 14 of clip 10, as was discussed with respect to FIGS. 12 and 13 above. Additionally, backstop clasps 20F and 20M are also engaged during actuation of clip 10 to further fixate the closure and keep clip 10 from reopening. Backstop clasp 20 is an improved feature of the present invention which provides additional security that is lacking in the prior art.

FIGS. 12-15 depict the vertical lengths of clasps 30F and 30M, including complementary ridges 90F and 90M, respectively, which enable clip 10 to provide secure retention of suture 1 within its body in center 14. Actuation of clip 10 also brings together foundations 100F and 100M, forming annulus 15 around recesses 80F and 80M. Annulus 15 distributes the forces generated by the secure fixation of suture 1 within upper body 40 evenly across tissue surface 3 upon which clip 10 rests. Additionally, recesses 80F and 80M form a space, i.e., quadrilateral 16, and allow stacking or nesting of multiple clips 10 if a further enhancement of security of clip 10 relative to suture 1 and/or tissue holding power is desired.

Turning now specifically to FIGS. 14 and 15, as was discussed above, forceps 2 may be used to actuate clip 10 to engage clasp 30 and backstop clasp 20. As is depicted in FIGS. 14 and 15, upper body 40F and 40M and footpads 50F and 50M provide surfaces for interaction with the jaws of forceps 2. In particular, the forceps jaws may be rested on and pressed against footpads 50F and 50M in order to actuate clip 10, e.g., the jaws of forceps 2 may be rested on footpads 50F and 50M in order to push the clip 10 posteriorly while holding the suture 1 anteriorly in order to maximize arterial pressure on the tissue surface 3. The forceps 2 may then actuate upper body 40F and 40M by clasping the clip 10 toward the vertical centerline 18 and securing the suture 1 in the center 14 of clip 10. Further, upper body 40F and 40M may be advantageously configured and dimensioned to include substantially flat vertical sides to provide a surgeon with a useful surface onto which forceps 2 may grip in order to position and actuate clip 10.

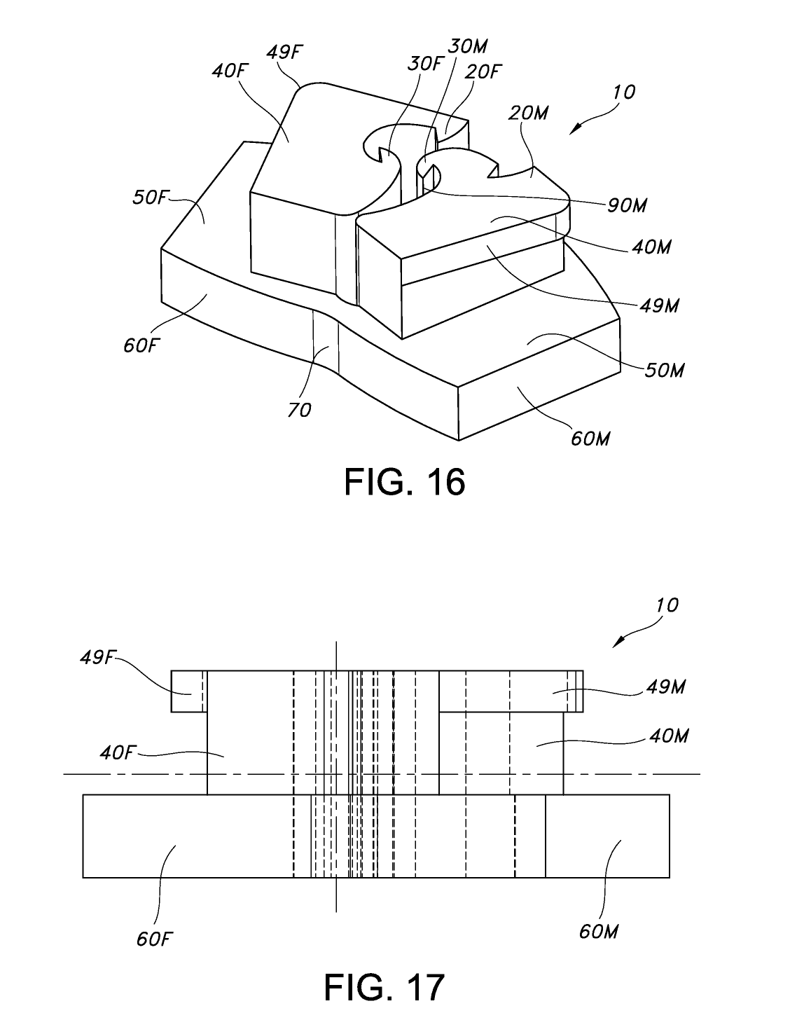

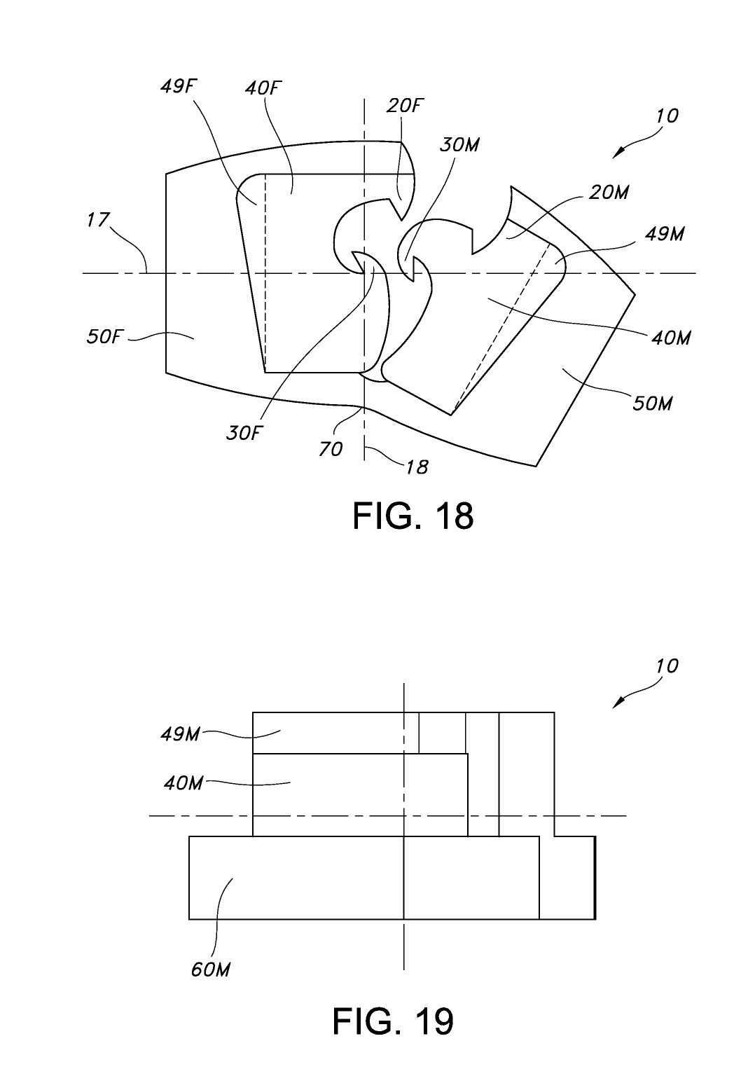

In an alternate exemplary embodiment of clip 10 illustrated in FIGS. 16-19, upper body 40F and 40M may define or contain small grasping ridges 49F and 49M, e.g., a “lip” or “ridge” protruding from the top portion of upper body 40F and 40M in order to provide an improved surface which would cooperate with forceps 2, thereby reducing the potential for forceps 2 to slide up and off from upper body 40F and 40M. As would be understood by one of ordinary skill in the art, each jaw of forceps 2 would fit between the footpads 50F and 50M and the grasping ridges 49F and 49M, respectively, on the highest surface of upper body 40F and 40M, thereby providing a stronger and more confident grip around clip 10 for actuation of clasp 30 and backstop clasp 20. Thus, the grasping ridges 49F and 49M would be configured and dimensioned in such a way as to provide sufficient support for forceps 2, while still permitting the stacking of additional clips 10, i.e., permitting upper body 40F and 40M to fit inside recesses 80F and 80M which form quadrilateral 16. Further, although illustrated as protruding in a graded manner, i.e., a smaller protrusion at the hinge 70 side and a greater protrusion at the side opposite the hinge 70 of upper body 40F and 40M, it should be understood that grasping ridges 49F and 49M can be configured and dimensioned to protrude at an even distance from the upper body 40F and 40M and can also include chamfered edges.

As has been discussed previously, installation/placement of suture clips of the prior art requires extra clip appliers, the use of both hands by the surgeon, and even assistance from another. Thus, a need exists for a suture clip which would provide efficient installation, as well as control for the surgeon during installation of the clip, without encountered the noted prior art limitations. The exemplary embodiments of clip 10 provide the surgeon with such control and autonomy, thereby meeting the existing needs. In particular, in addition to being used with standard trocars and standard forceps, clip 10 can be inserted, actuated and fixated by the surgeon without the need for an assistant. To do so, referring to FIGS. 12-15, the surgeon uses endoscopic forceps 2 to insert clip 10 into the surgical field. The surgeon then draws suture 1 into the void between clasps 30F, 30M and backstop clasp 20F to hook or otherwise position clip 10 relative to suture 1. When suture I is pulled anteriorly, clip 10 drops to tissue surface 3, where it is already in close approximation to its final location. Also by design, clip 10 is actuated posteriorly from overhead by the surgeon directly, instead of medially by the surgical assistant. To do so, the surgeon pulls suture 1 taut anteriorly and sets endoscopic forceps 2 onto footpads 50. The surgeon then pushes posteriorly (down) on clip 10 to induce maximum arterial pressure within the organ, and then actuates upper body 40F and 40M in this pressurized position directly. During actuation of clip 10, clasps 30 and backstop clasps 20 are engaged to hold suture 1 and the body of clip 10 securely locked. This process is subsequently repeated as necessary for each clip 10 the surgeon wishes to apply. Through these design elements, clip 10 provides surgeons the convenience and autonomy they greatly desire and that which is lacking in the prior art.

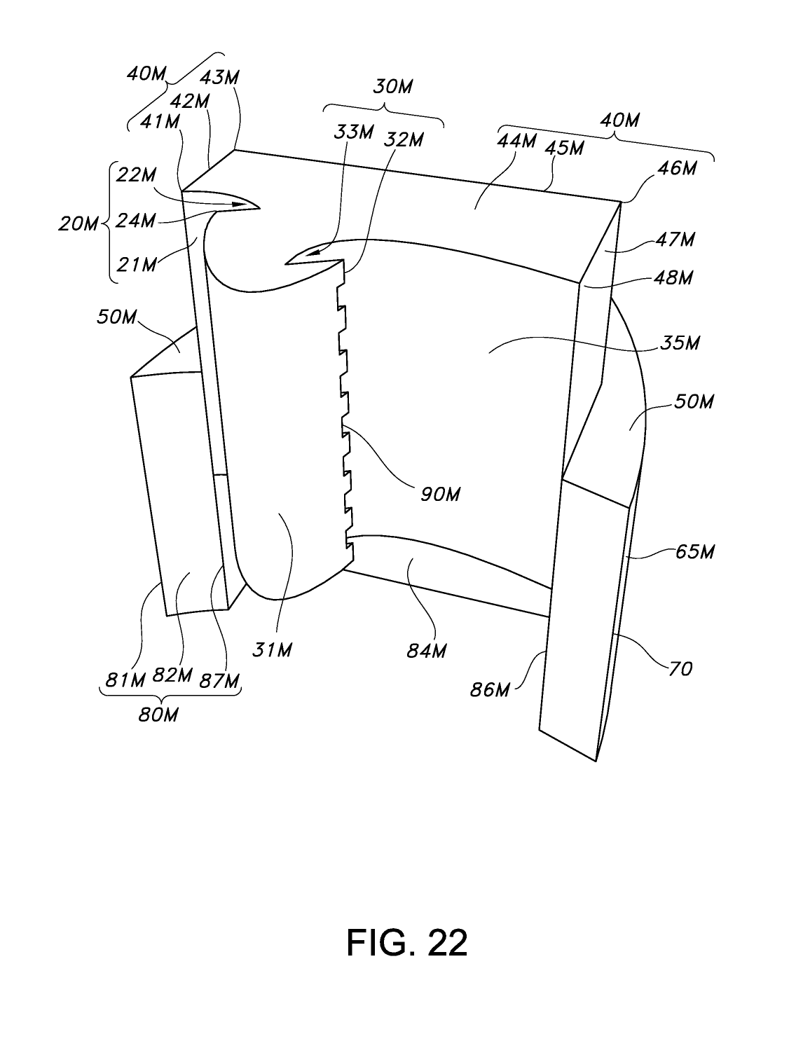

A suture clip's security, holding strength and surface pressure are fundamental to its utility in a surgical procedure. The exemplary embodiments described herein of clip 10 provide significant advantages over existing surgical slips in this regard. Unlike suture clips taught by the prior art, clip 10 includes two (2) pairs of clasps, i.e., clasps 30 and backstop clasp 20, in order to establish an extremely secure closure. Per FIGS. 12-15, clasp 30 concentrates on holding suture 1 in a specific locked position, specifically the region/crevice at center 14 of clip 10. As clip 10 is actuated toward vertical centerline 18 depicted in FIG. 13, clasp 30M is deformed inward as it traverses clasp 30F via its rounded clip surface 31F. (Sub-features of clasps 30 and 20 are delineated for the “male” side IOM and “female” side 1OF in FIGS. 22-25 and FIGS. 26-29, respectively.) As clasp 30M continues past apex 33F, clasp 30M drives suture 1 with its leading edge 32M and ridges 90M into crevice 32F, fixating the position of suture 1 at center 14. This accurate positioning of suture 1 at center 14 is further highlighted in orthogonal views represented in FIGS. 12 and 13.

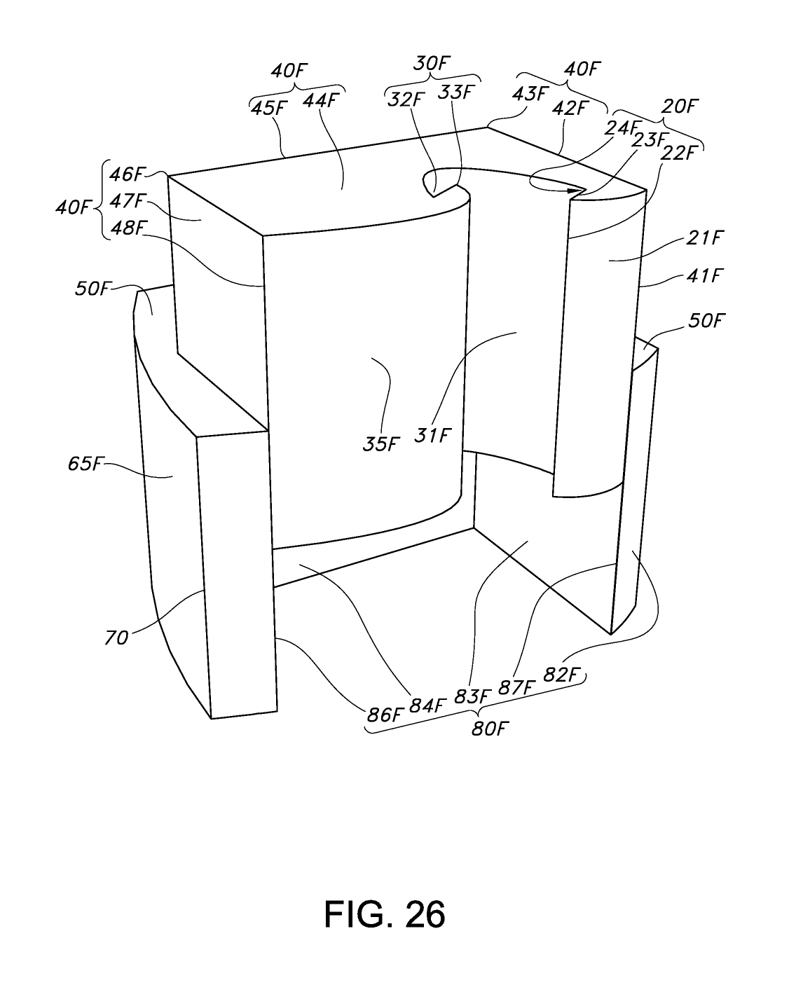

Still with reference to FIGS. 12-15, whereas clasp 30 focuses on fixating suture 1, backstop clasp 20 supplements the holding power of clasp 30 by establishing a secure backstop to help keep clip 10 closed throughout the patient's complete wound-healing period. Especially acute periodic events, e.g., patient coughing, seizure, trauma and the like, can potentially exert significant forces on a clip that may compromise its integrity and function post-operation. Nonetheless, through its design, backstop clasp 20 of clip 10 provides an additional level of security to help clip 10 endure through these types of events. Specifically, as clip 10 is actuated toward vertical centerline 18, as shown in FIGS. 12-15 and in greater detail in FIGS. 26-29, leading edge 22F is deformed inward as it traverses rounded clip surface 31M. As it continues past edge 24M at the distal end of surface 31M, leading edge 22F releases from its deformation to lock into crevice 22M. Unlike edges 32F and 32M, edges 22F and 22M do not contain ridges similar to 90F and 90M, because there is no suture 1 to hold. Rather, backstop clasps 20F and 20M establish a backstop for the present clip 10 through mating of surfaces 21F with 21M, 23F with 23M, and 82F with 82M for lower body 60.

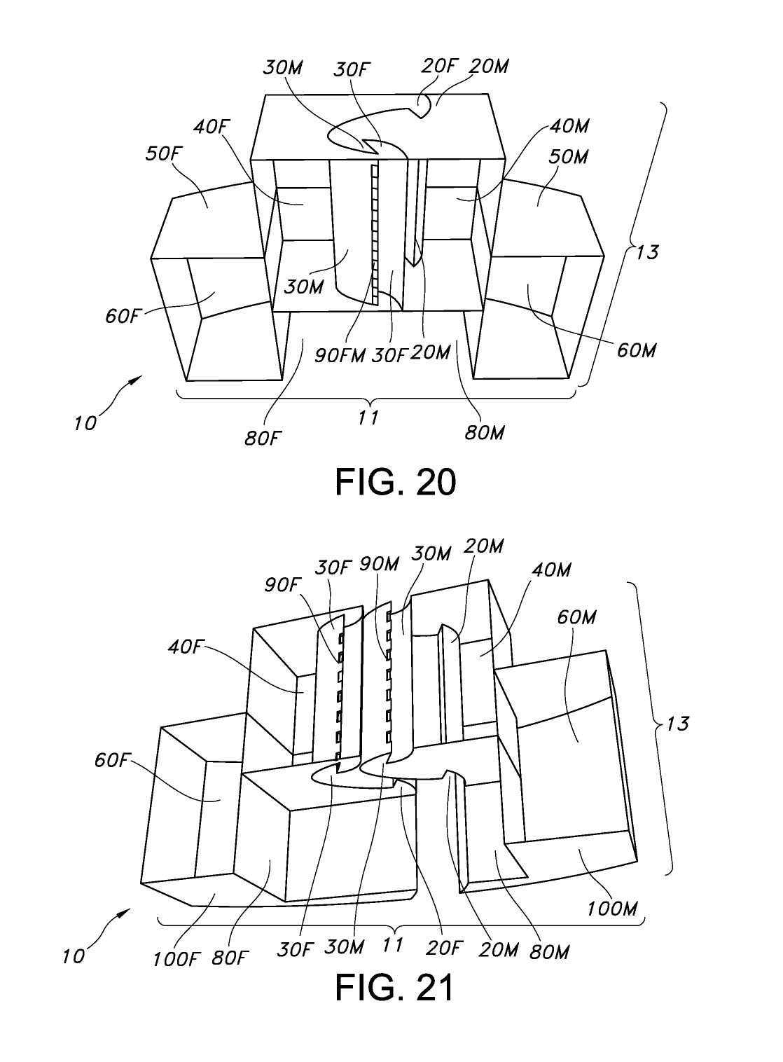

Turning now to FIGS. 20 and 21, cross-sectional views are provided of the top and bottom of exemplary clip 10 in a closed and open position, respectively. It should be noted that FIGS. 20 and 21 are illustrated in a transparent manner to provide a better view of the details of the exemplary clip 10 and, thus, should not be interpreted as portraying a hollow exemplary clip 10. Specifically, FIG. 21 shows a clear representation of clip 10 in an open position prior to actuation, wherein complimentary ridges 90F and 90M formed or otherwise located on clasp 30F and 30M—which capture and secure suture 1—can be seen. Once clip 10 is actuated by forceps 2, clasp 30F and 30M and backstop clasp 20F and 20M, as well as ridges 90F and 90M, engage and provide a strong holding force relative to suture 1. The closed clip 10 can be seen in FIG. 20, specifically with respect to the engaged clasp 30 and backstop clasp 20. Ridges 90F and 90M are configured and dimensioned to fit inside each other, i.e., to interdigitate, in order to provide a strong and secure hold relative to suture 1.

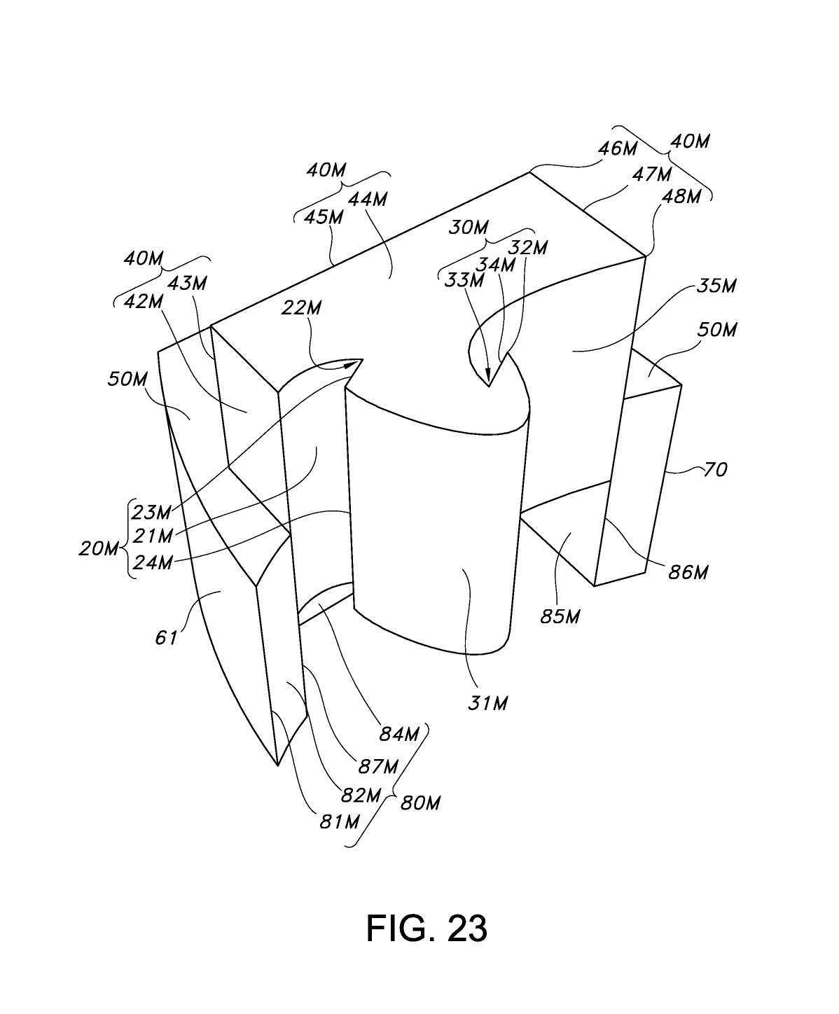

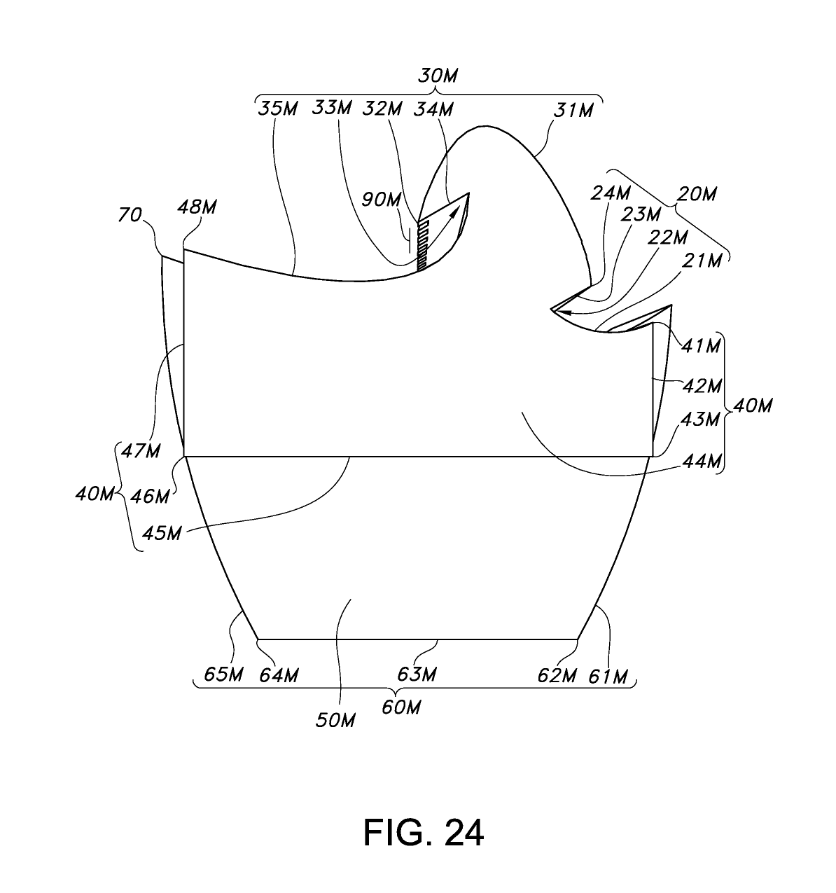

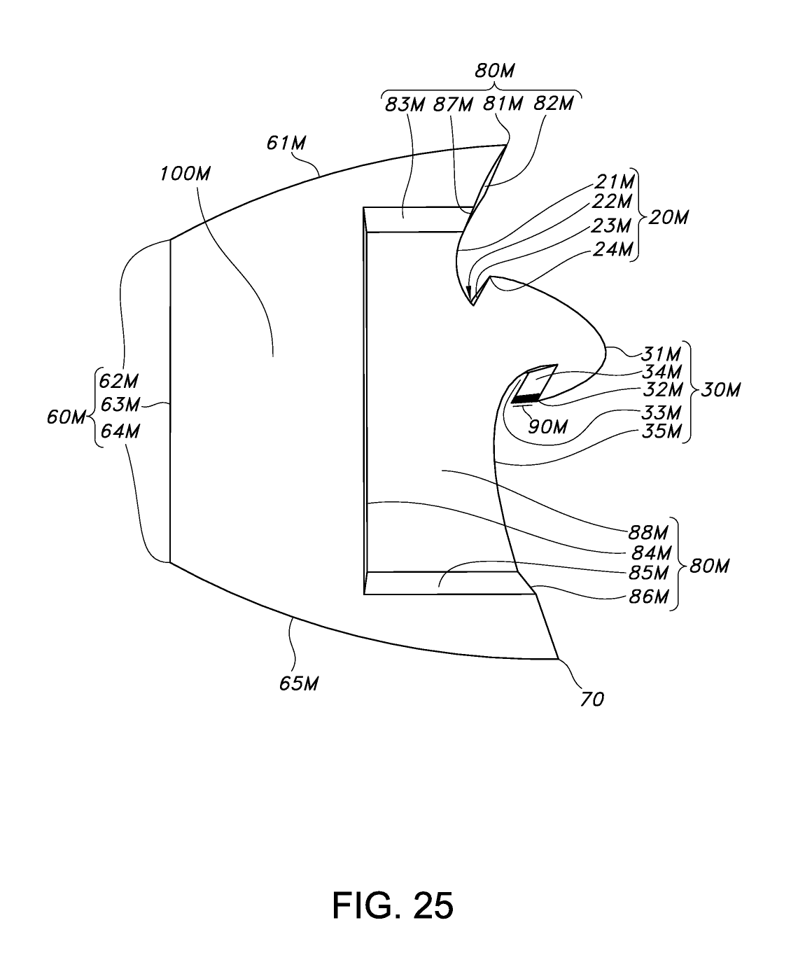

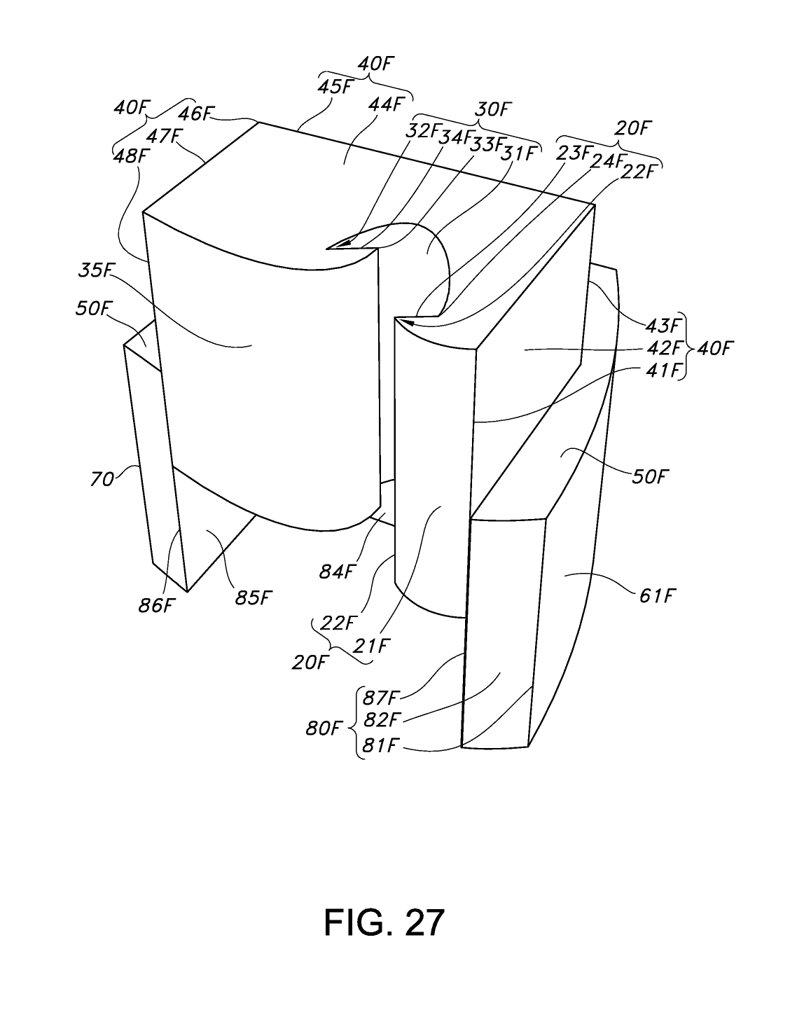

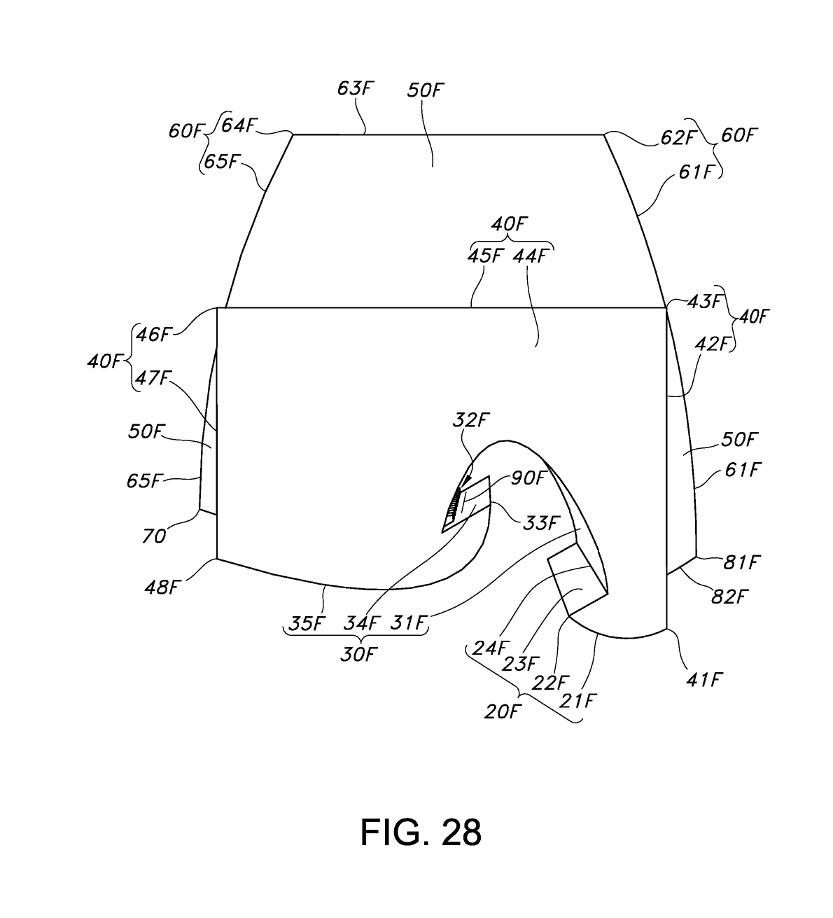

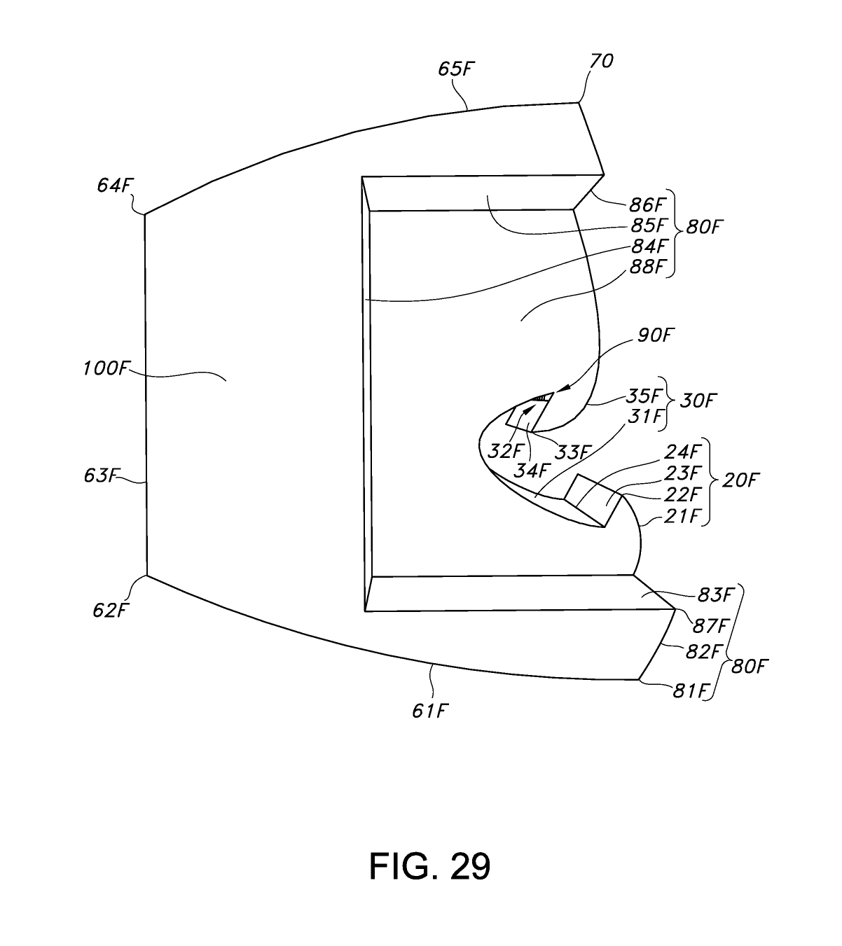

FIGS. 22-25 show detailed perspective views of the “male” leg/side 10M of clip 10. Specifically, FIG. 22 depicts the “male” hinge 70 side of clip 10, FIG. 23 depicts the “male” leg/side 10M opposite of hinge 70, FIG. 24 depicts a top view of the “male” leg/side 10M and FIG. 25 depicts a bottom view of the “male” leg/side 10M of clip 10. Similarly, FIGS. 26-29 show detailed perspective views of the “female” leg/side 10F of clip 10. Specifically, FIG. 26 depicts a view of the “female” hinge 70 side, FIG. 27 depicts a perspective view of the “female” leg/side 1OF opposite of hinge 70, FIG. 28 depicts a top view of the “female” leg/side 10F, and FIG. 29 depicts a bottom view of “female” leg/side 10F of clip 10. All of the components and structural features of the “male” leg/side 10M and “female” leg/side 10F of clip 10 which have been discussed above are depicted in greater detail in FIGS. 22-29. The details of both the “female” leg/side 10F and “male” leg/side IOM are substantially similar. Specifically, clasp 30 includes the sub-components/features of surface 31, apex 33 and edge 32. Backstop clasp 20 includes the sub-components/features of surface 21, edge 22, and edge 24. Upper body 40 and lower body 80 further include sub-components/features, i.e., surfaces, 41, 42, 43, 44, 45, 46, 47, 48 and 81, 82, 84, 86 and 87, respectively.

With reference again to FIGS. 12 and 13, annulus 15 on the posterior surface, i.e., foundation 100F and 100M, of clip 10 represents the projected line of force distribution that is presented to tissue surface 3 upon which clip 10 rests. Specifically, force distribution of suture 1 is transmitted conically from suture 1, through clasp 30, upper body 40, lower body 60, and foundation 100 to tissue surface 3. Although the force distribution of annulus may be circular, the exemplary clip 10 has outer dimensions 11, 12 and 13, which are designed to incorporate into the surgical area by fitting clip 10 through a desired port, e.g., an approximately 8 mm or larger trocar port. Thus, as represented in FIG. 13, clip 10 and annulus 15 are elongated into a substantially elliptical form, with horizontal centerline 17 as its major axis. The comparable force distribution area for existing surgical clips as taught by the prior art is much smaller, unevenly distributed and inconsistently positioned.

In the present disclosure, the force distribution of annulus 15 of clip 10 is significant, because it distributes the force of suture 1 evenly and over a larger surface area. For example, a kidney's parenchymal tissue is extremely soft, such that a suture can tear through it quite easily without adequate force distribution. While the existing clips in the prior art provide some force distribution, patient complications have shown that they are not adequate enough in all situations. In comparison, center 14 of clip 10 and its corresponding annulus 15 greatly enhance the present invention's ability to distribute the force of suture 1 evenly, precisely and over a larger surface area, potentially reducing the risk for patient complications.

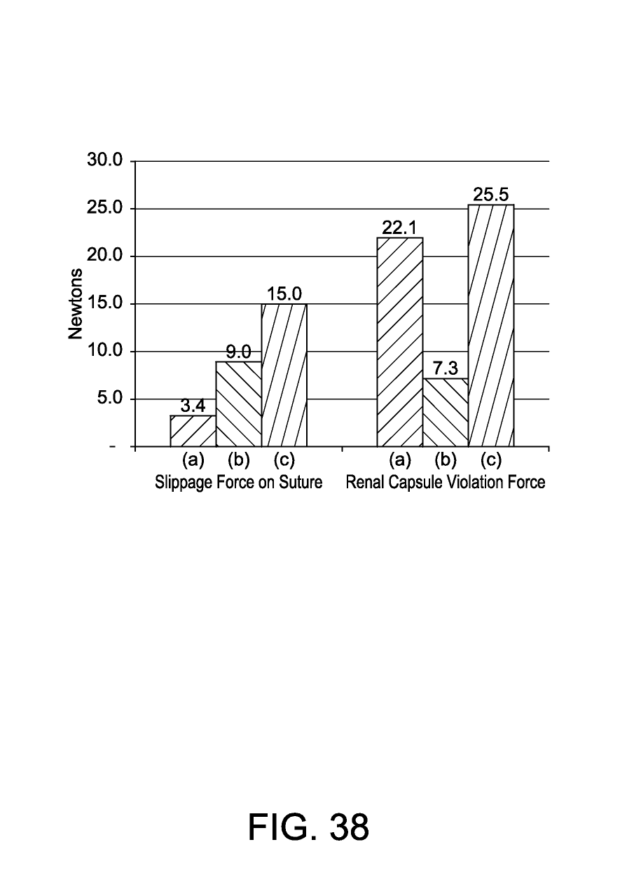

FIG. 38 is a bar chart representing the improved strength and force distribution of the disclosed exemplary clip 10 with respect to the prior art. Specifically, FIG. 38 provides comparative strength profiles of clip 10 with respect to the Hem-O-Lok™ and Lapra-Ty™ clips, prior art clips discussed above in relation to FIG. 1. The Hem-O-Lok™ and Lapra-Ty™ clip values are based on prior industry studies (see, e.g., Tarin, T. et al., Comparison of Holding Strength of Suture Anchors on Human Renal Capsule, Journal of Endourology, Vol. 24, No. 2, p. 293-297 (2010)), while the exemplary clip 10 values are based on, e.g., the design of the clip 10, the expected surface tension, the renal capsule violation force based on the geometry of the clip 10, and the like, and do not include the effects of anti-backup features discussed previously. It should therefore be understood that FIG. 38 is provided for comparison purposes and should not be taken as a representation of the full potential of the exemplary clip 10 in, e.g., retaining sutures, providing a renal capsule force, or the like.

The first group of “bars” in FIG. 38 represents the data for slippage force on suture 1, while the second group of “bars” represents the data for the renal capsule violation force, i.e., the force on tissue surface 3. In each group of “bars”, bar (a) is the force for the Hem-O-Lok™ clip, bar (b) is the force for the Lapra-Ty™ clip, and bar (c) is the force for the presently disclosed clip 10. As can be seen from the data in FIG. 38, the improved design of clip 10 provides the highest, and thus strongest, slippage force on suture 1 in combination with the renal capsule violation force. With respect to bar (a) of the Hem-O-Lok™ clip, although the renal capsule violation force may be strong, it is still lower in force than that of the disclosed clip 10, and because the slippage force on suture 1 of the Hem-O-Lok™ clip is extremely low, the Hem-O-Lok™ clip requires additional security from the implementation of at least secondary clips and possibly more. Thus, the Hem-O-Lok™ clip cannot provide the same renal capsule violation force in combination with the slippage force on suture 1 as the disclosed clip 10, whether it is implemented on its own or in combination with a secondary clip. In addition, the need for at least secondary clips when utilizing the Hem-O-Lok™ clip complicates the surgical procedure by the amount of time, e.g., the surgical time generally and/or the warm ischemia time, and equipment, and thus money, required to secure suture 1. Similarly, the data corresponding to the Lapra-Ty™ clip, bar (b), depicts a low slippage force on suture 1 and an even lower renal capsule violation force. Thus, the Lapra-Ty™ clip cannot meet the high strength profiles of the disclosed clip 10, which offers about a 15 N slippage force on suture 1 and about a 25.5 N renal capsule violation force. As described earlier, the design of clip 10 thus provides a higher and more evenly distributed force on, e.g., the renal capsule, as well as retaining suture 1 in a way which substantially reduces slippage. Further, it should be noted that the clip 10 can be implemented in a variety of surgical applications, e.g., renal, hepatic, and the like.

As was discussed previously, sutures may be composed in a variety of forms, including monofilament and braided sutures. Specifically, monofilament sutures are composed of a single filament and generally are more likely to slip or pass through a clip, especially in a wet surgical environment. On the other hand, braided sutures are composed of two or more filaments and provide greater friction on braided surfaces to reduce the amount of slip or passage through a clip. However, due to the roughness of the surface of the braided suture, greater inflammation and damage to tissue generally results as the braided suture is passed through the tissue. In particular, the braided surface of the suture can act similarly to a serrated edge, thereby causing tearing and/or tissue trauma. As currently utilized in the industry, suture clips of the prior art fail to provide sufficiently high forces on sutures to prevent slippage. Therefore, prior art suture clips may generally only be implemented with a braided suture, which provides a rougher surface to decrease the amount of slippage. Particularly, as can be seen from the slippage forces on a braided suture 1 with respect to the prior art in FIG. 38, the Hem-O-Lok™ and Lapra-Ty™ clips offer significantly lower slippage forces on the suture in comparison to clip 10. On the other hand, because clip 10 offers a significantly higher slippage forces on the suture, the presently disclosed clip 10 may be utilized with a monofilament and/or braided suture, as desired by the surgeon. The improved force generated by clip 10 thereby provides more flexibility for the surgeon performing the operation and permits the use of monofilament sutures, which may reduce the amount of tissue trauma and recovery time for a patient.

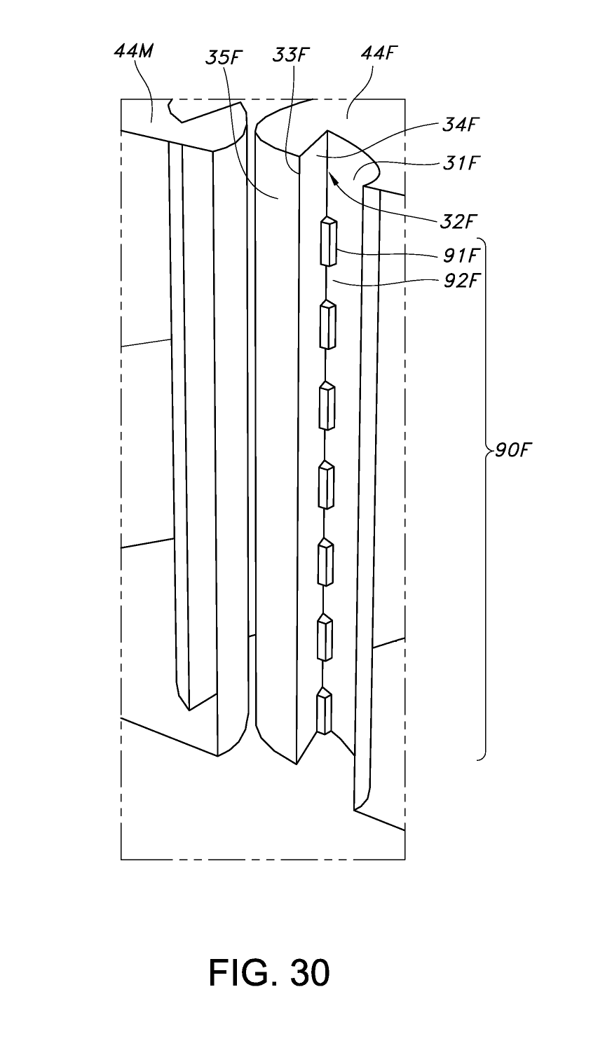

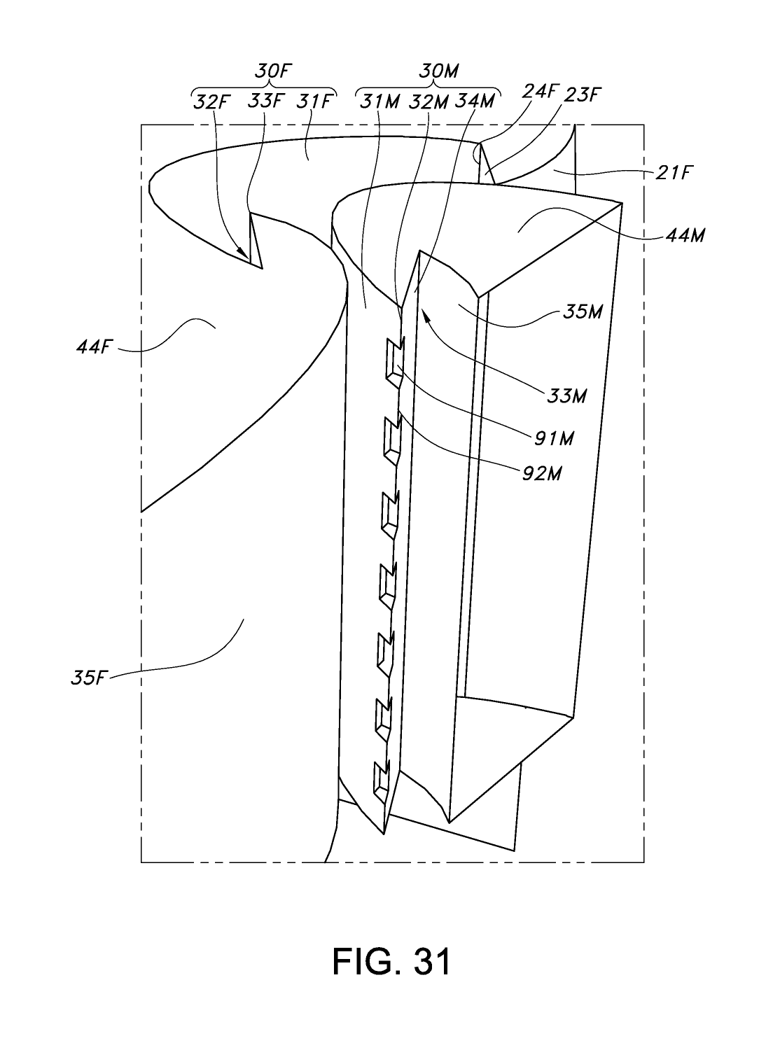

The ridges 90F and 90M discussed above with respect to FIGS. 20 and 21 are depicted in greater detail in FIGS. 30 and 31. Specifically, ridges 90F and 90M traverse the lengths of edges 32F and 32M, respectively. As clip 10 is actuated via forceps 2, ridges 90F and 90M interlock with each other, forming a tongue and groove joint 90FM and establishing a substantially strong hold on suture 1. Further, each female tongue 91F protrudes to drive suture 1 into complementary male groove 91M. Conversely, each male tongue 92M also protrudes to drive suture 1 into a complementary female groove 92F. Blood vessels have diameters of approximately 5.6 mm and, because the existing surgical clips as taught by the prior art were initially designed to ligate blood vessels, they have clasps that are much too large to accurately position and hold a suture 1 in a precise location. In contrast, the tongue and groove joints of the present clip 10 are designed to specifically fixate a variety of suture sizes, e.g., 1, 0, 2/0, 3/0 and 4/0, in a precise location at center 14. These suture sizes have significantly smaller diameters than blood vessels, ranging only from approximately 0.4 mm to 0.15 mm, respectively. Accordingly, protrusions of male tongues 91M and female tongues 92F, depths of female grooves 91F and male grooves 92M, width of male edge 32M and width of female crevice 32F each measure less than about 0.5 mm, thereby accommodating a design specifically made to fixate suture 1 and provide the greatest holding strength on suture 1.

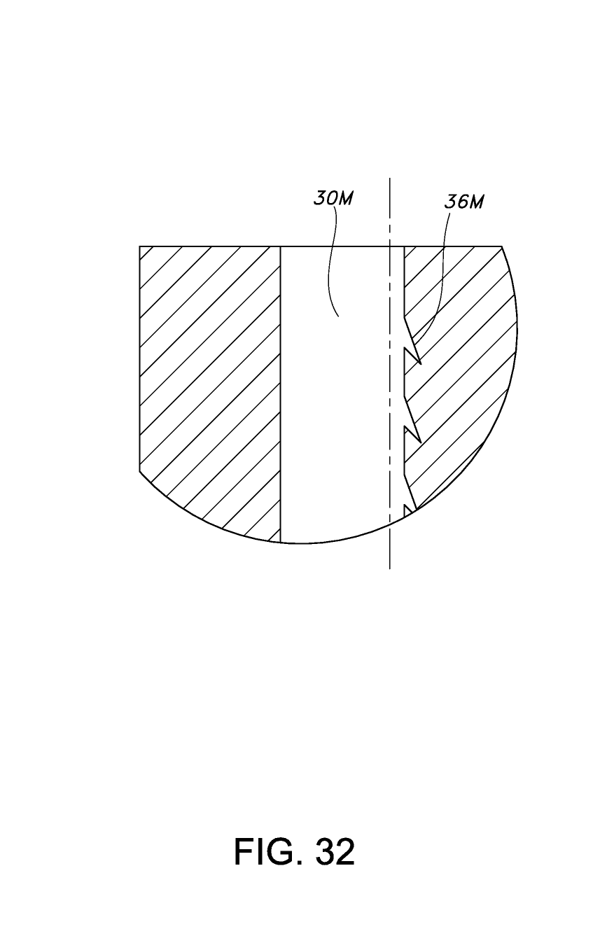

In an alternate exemplary embodiment of clip 10 illustrated in FIG. 32, in order to further improve the slippage force on a suture, anti-backup features 36F (not shown) and 36M may be implemented on the inside surfaces of clasp 30F and/or 30M, separately or in conjunction with the previously discussed suture retention features, to engage the suture 1, Although FIG. 32 illustrates the exemplary anti-backup feature 36M on the inside surface and/or leading edge of clasp 30M, it should be understood that a substantially similar anti-backup feature 36F may be implemented on clasp 30F. The anti-backup feature 36M may include an inwardly directed notch, e.g., a ratchet, and/or a rough surface to increase the friction force on the suture. If an inwardly directed notch is implemented, the notch is generally configured and dimensioned in such a way as to permit clip 10 to be actuated around suture 1 and to further permit clip 10 to be moved along suture 1 in a single direction only. As would be understood by one of ordinary skill in the art, the inwardly directed anti-backup feature 36M would allow a surgeon to actuate and slide clip 10 in one direction against tissue surface 3 along suture 1 as desired, while the anti-backup feature 36M would engage suture 1 in such a way as to prevent clip 10 from sliding away from tissue surface 3. Further, the anti-backup feature 36M, e.g., the inwardly directed notch, can be a single-strand, unidirectional notch, can restrain the clip 10 from backing out from its position relative to the suture 1, and can be configured and dimensioned appropriately depending on the suture 1 size being implemented. In addition, the monofilament and/or braided suture 1 can be positioned in the clip 10 at the location designated by the vertical axis. A rough surface may also be implemented, whether in combination with or separate from inwardly directed anti-backup feature 36M. The rough and/or abrasive surface may be composed of ridges, spikes or the like, thereby creating a higher friction force on suture 1 as it is engaged between clasp 30F and 30M. Thus, an even higher slippage force on suture 1 may be supplied for a more secure hold of either a monofilament or a braided suture.

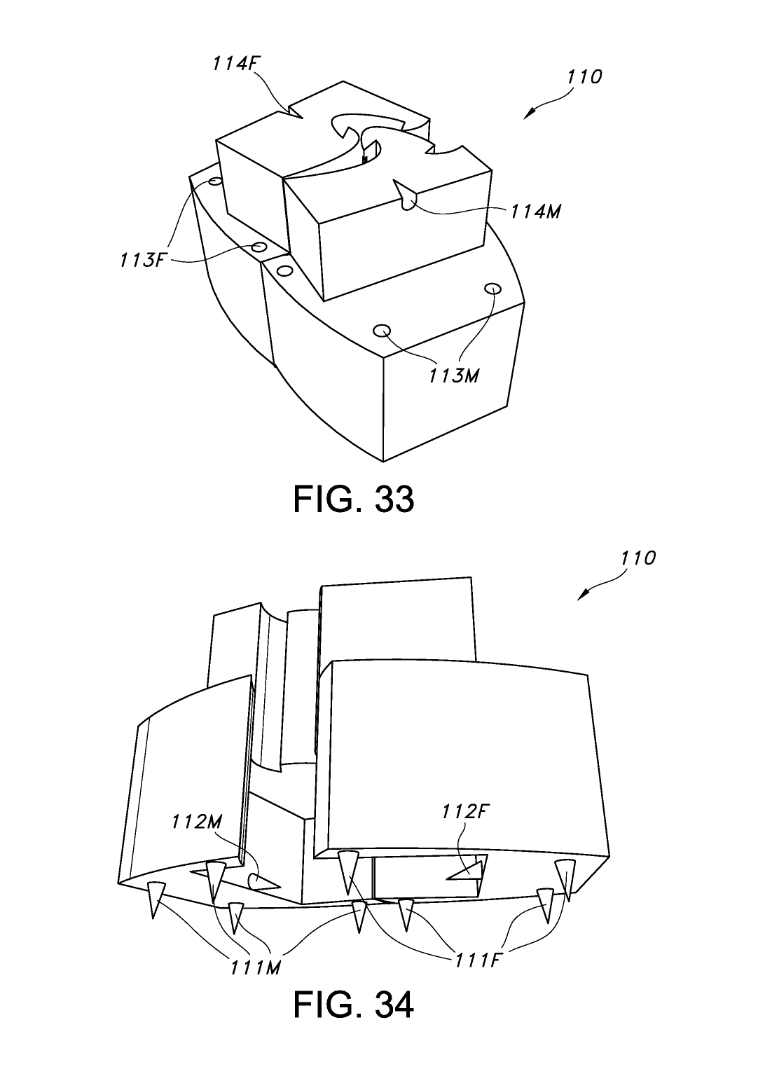

With reference to FIGS. 33 and 34, another exemplary embodiment of clip 110 is depicted. Clip 110 is composed of substantially similar components as were discussed with respect to clip 10, except for the changes discussed below. Specifically, additional features, i.e., grip points, spikes and the like, may be implemented in order to enhance the characteristics of clip 10 discussed above. As can be seen in FIGS. 33 and 34, a number of grip point feet 111F and 111M around the perimeter of foundation 100 may be implemented. It should be noted that the embodiments depicted in FIGS. 33 and 34 are merely exemplary representations of clip 10 and, therefore, do not create limitations of the grip points depicted. Particularly, the grip point feet 111F and 111M may or may not be as sharp or of the dimensions as presented herein, but their function would be the same as that proposed, namely to provide increased holding power of clip 10 on tissue surface 3. Tissue piercing points 112F and 112M are also depicted in underside recess 80 of clip 10. These piercing points 112F and 112M are included to pierce through tissue surface 3 and facilitate fixation of clip 10 in an orthogonal direction and prevent its migration. Tissue piercing points 112F and 112M may further be angled posteriorly to facilitate improved piercing and fixation through tissue surface 3. Further, recesses 113F, 113M, 114F and 114M are of the inverse design of tissue grip points 111 and tissue piercing points 112 in order to enable stacking of multiple units of clip 10, if desired.

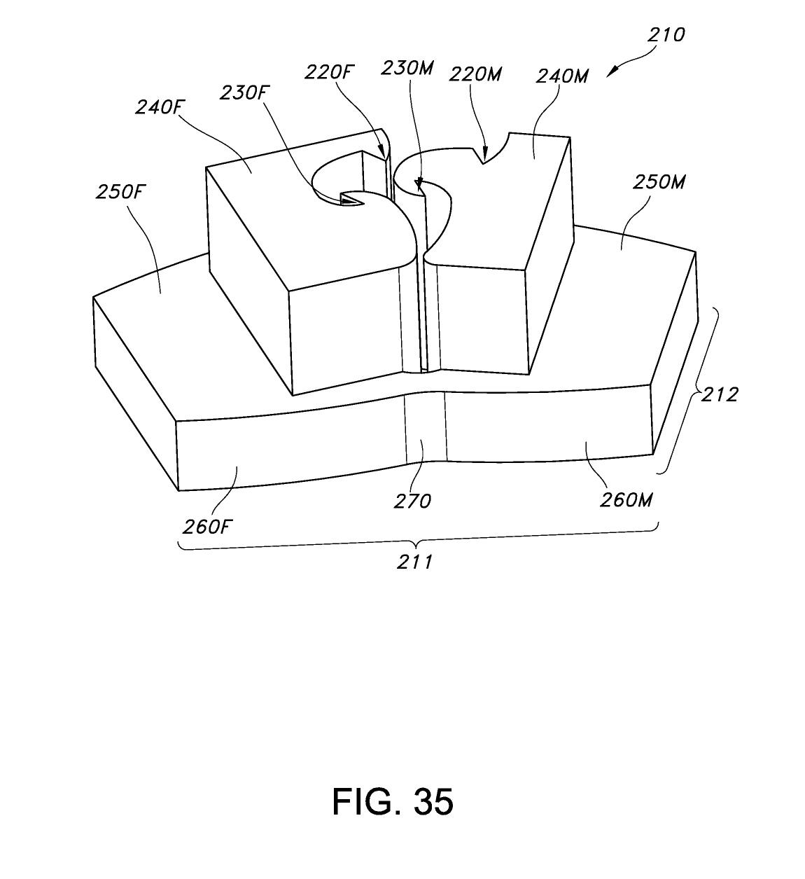

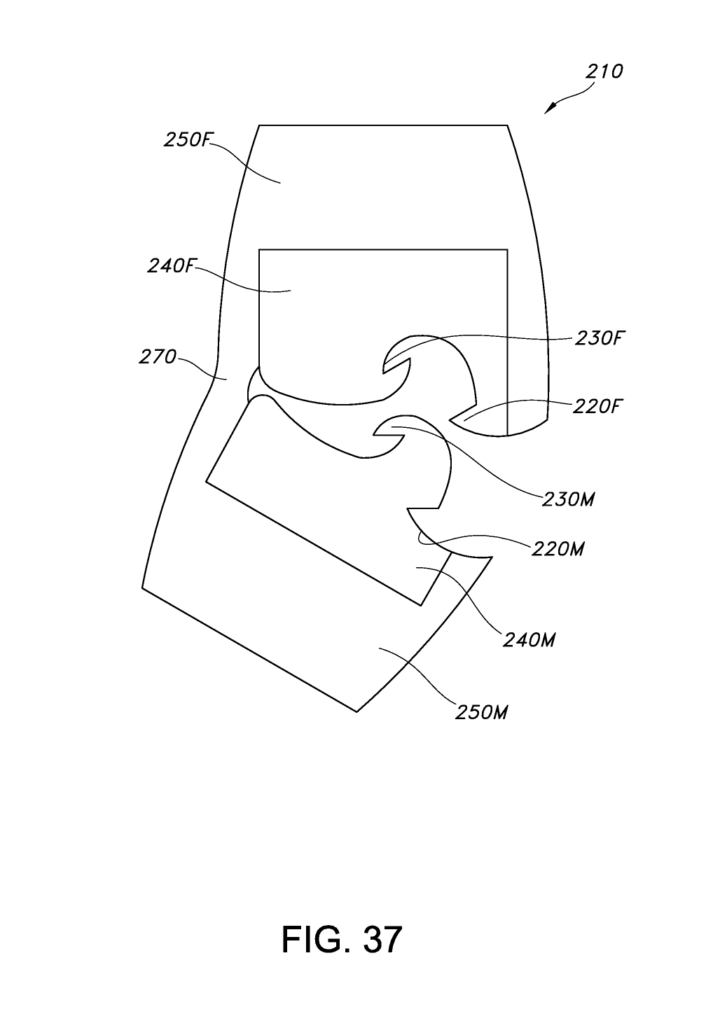

Turning now to FIGS. 35-37, another exemplary embodiment of clip 210 is depicted in accordance with the present disclosure. Clip 210 is composed of substantially similar components as were discussed with respect to clip 10, except for the changes discussed below. Specifically, clip 210 includes a living hinge 270, which may be configured and dimensioned to have a rounded and/or smoothly transitioned and continuous form at the axis of rotation, rather than the angled form of hinge 70 of clip 10. Although not limited to the configuration or dimensions of hinge 270 as depicted in FIGS. 35-37, the exemplary hinge 270 provides a means for reducing stress at the joint between the “female” side 210F and “male” side 210M of clip 210 by e.g., replacing the angled and/or highly curved features of the hinge with a continuous and/or smooth transition between the “female” side 210F and “male” side 210M. By implementing a rounded living hinge 270, the duration and effectiveness of clip 210 may be substantially improved due to the longer-lasting and smoother ductility of hinge 270. Further, as depicted in FIGS. 35-37, upper body 240F and 240M may also include a chamfered inner edge on the side closest to the center of clip 210 and located along the hinge 270 axis of rotation. The configuration and dimensions of the chamfered edge of upper body 240F and 240M shall not be limited to that shown in FIGS. 35-37, but may instead be implemented as desired by those skilled in the art in order to provide a smoother engagement of clasp 230 and backstop clasp 220, thereby creating a more efficient actuation of clip 210.

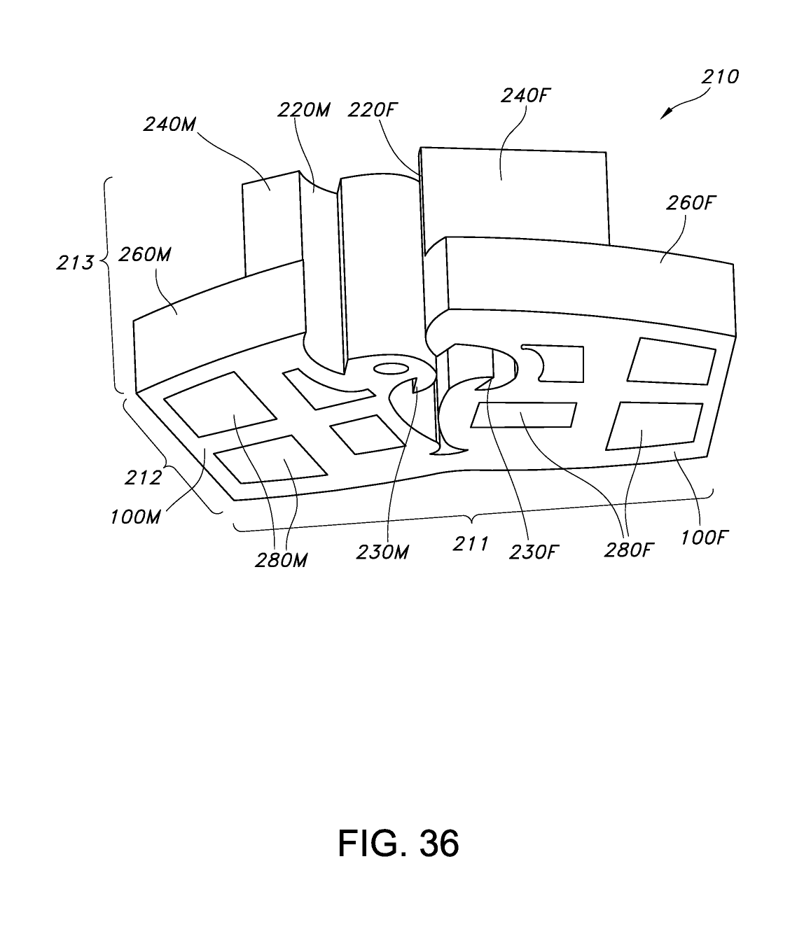

Now turning specifically to FIG. 36, a perspective view of the exemplary embodiment of clip 210 is depicted in accordance with the present disclosure. Specifically, clip 210 may include numerous recessed undersides 280M and 280F. The multiple recessed underside structure of clip 210 may further provide a substantially stronger clip 210 due to the extra support from foundation 100F and 100M members crossing the lower body 260F and 260M, respectively. The implementation of the lower body 260 in FIG. 36 may additionally create a substantially more evenly distributed force on tissue surface 3 due to more evenly distributed surface area of foundation 100F and 100M which will be applied directly onto tissue surface 3. Further, the recessed undersides 280M and 280F can hasten the absorption properties of the material, thereby reducing the time for recovery and potential post-operation complications generally found to occur with clips taught by the prior art, e.g., formation of kidney stones, blocked ureters, and the like.

Although the present disclosure has been described with reference to exemplary embodiments and implementations, it is to be understood that the present disclosure is neither limited by nor restricted to such exemplary embodiments and/or implementations. Rather, the present disclosure is susceptible to various modifications, enhancements and variations without departing from the spirit or scope of the present disclosure. Indeed, the present disclosure expressly encompasses such modifications, enhancements and variations as will be readily apparent to persons skilled in the art from the disclosure herein contained.

1. A surgical clip, comprising:
a. a first leg and a second leg in opposed relation to the first leg;
b. a flexible hinge integrally disposed between and joining the first leg and second leg;
c. a first pair of cooperating locking members defined on the first and second legs; and
d. a second pair of cooperating locking members defined on the first and second legs;
wherein flexibility associated with the flexible hinge permits the first and second legs to be brought into close approximation, thereby permitting interlocking engagement of the first pair of cooperating locking members and interlocking engagement of the second pair of cooperating locking members.

2. The surgical clip of claim 1, wherein the first pair of cooperating locking members include first cooperating male and female locking members defined on the first and second legs, respectively, and wherein the second pair of cooperating locking members include second cooperating male and female locking members defined on the first and second legs, respectively.

3. (canceled)

4. The surgical clip of claim 2, wherein at least one of the first pair of cooperating locking members and the second pair of cooperating locking members include surface characteristics for providing improved suture retention when the first and second legs are brought into close approximation.

5. The surgical clip of claim 4, wherein the surface characteristics are ridges, and wherein the ridges are included on at least one of the first and second legs, and wherein the ridges on the first leg are configured to be interdigitated with the ridges on the second leg when the first and second legs are brought into close approximation.

6. (canceled)

7. The surgical clip of claim 1, wherein the first and second legs define first and second lower body regions, wherein the first and second body regions define recessed undersides, and wherein the recessed undersides are configured and dimensioned to permit stacking or nesting of multiple surgical clips when placed in close approximation.

8. (canceled)

9. The surgical clip of claim 1, wherein the first and second legs define first and second lower body regions, wherein the lower body regions define forceps footpads, and wherein the forceps footpads are configured and dimensioned for cooperation interaction with forceps jaws.

10. (canceled)

11. The surgical clip of claim 1, wherein the flexible hinge defines a substantially angled configuration.

12. The surgical clip of claim 1, wherein the flexible hinge defines a substantially circular configuration.

13. The surgical clip of claim 1, wherein at least one of the first leg and the second leg defines an anti-backing structure that is configured and dimensioned to engage a suture positioned between the first and second legs to permit movement of the suture in a first direction, but to substantially prevent movement of the suture in a direction opposite to the first direction, and wherein the anti-backing structure is selected from the group consisting of an inwardly-directed notch and one or more inwardly-directed surface protrusions.

14. (canceled)

15. The surgical clip of claim 13, wherein the anti-backing structure is effective for control of movement of a monofilament suture.

16. The surgical clip of claim 1, wherein the surgical clip is fabricated from a biocompatible material.

17. The surgical clip of claim 1, wherein the first pair of cooperating locking members include a first locking member and a second locking member, and wherein the first and second locking members are adapted to receive and engage a suture in a suture capture region defined therebetween.

18. The surgical clip of claim 17, wherein the first locking member is a male locking member and the second locking member is a female locking member, and wherein the suture capture region is effective to retain a suture in a substantially centered position relative to the surgical clip.

19. (canceled)

20. A method for surgical clip application, comprising:
a. introducing a surgical clip in an open position into a surgical environment, wherein the surgical clip includes: (i) a first leg and a second leg in opposed relation to the first leg; (ii) a flexible hinge integrally disposed between and joining the first leg and second leg; (iii) a first pair of cooperating locking members defined on the first and second legs; and (iv) a second pair of cooperating locking members defined on the first and second legs;
b. positioning the surgical clip in the open position such that a suture in the surgical environment is positioned between the first leg and the second leg of the surgical clip; and
c. securing the suture between the first leg and the second leg of the surgical clip by moving the first leg and the second leg from the open position into close approximation, thereby causing interlocking engagement of the first cooperating locking members and interlocking engagement of the second cooperating locking members.

21. The method of claim 20, further comprising moving the surgical clip in a first direction along the suture without disengagement of the first cooperating locking members or the second cooperating locking members, and wherein the surgical clip includes a structural feature that substantially prevents movement of the surgical clip in a direction opposite to the first direction.

22. The method of claim 20, further comprising moving the surgical clip in a first direction along the suture prior to engagement of the first cooperating locking members or the second cooperating locking members.

23. (canceled)

24. The method of claim 20, further comprising applying at least one additional surgical clip to the suture, wherein the surgical clip and the at least one additional surgical clip include structural features that facilitate stacking or nesting thereof on the suture.

25. A method for surgical clip application, comprising:
a. positioning a surgical clip in an open position such that a suture is positioned between the first leg and the second leg of the surgical clip, wherein the surgical clip includes: (i) a first leg and a second leg in opposed relation to the first leg; (ii) a flexible hinge integrally disposed between and joining the first leg and second leg; (iii) a first pair of cooperating locking members defined on the first and second legs; and (iv) a second pair of cooperating locking members defined on the first and second legs;
b. securing the suture between the first leg and the second leg of the surgical clip by moving the first leg and the second leg from the open position into close approximation, thereby causing interlocking engagement of the first cooperating locking members and interlocking engagement of the second cooperating locking members; and
c. introducing the surgical clip into a surgical environment.

26. The method of claim 25, further comprising moving the surgical clip in a first direction along the suture without disengagement of the first cooperating locking members or the second cooperating locking members, wherein the surgical clip includes a structural feature that substantially prevents movement of the surgical clip in a direction opposite to the first direction.

27. (canceled)

28. The method of claim 25, further comprising applying at least one additional surgical clip to the suture, wherein the surgical clip and the at least one additional surgical clip include structural features that facilitate stacking or nesting thereof on the suture.
